SEQUENCE LISTING (19 sequences)

SEQ ID NO: 1 - DNA, 25 bp, Artificial Sequence
  STAT1 oligonucleotide
caccgggtggcaaatgaaacatcat

SEQ ID NO: 2 - DNA, 25 bp, Artificial Sequence
  STAT1 oligonucleotide
caccggaggtcatgaaaacggatgg

SEQ ID NO: 3 - DNA, 25 bp, Artificial Sequence
  STAT1 oligonucleotide
caccgcaggaggtcatgaaaacgga

SEQ ID NO: 4 - DNA, 25 bp, Artificial Sequence
  NTC oligonucleotide
caccgaaccggatcgccacgcgtcc

SEQ ID NO: 5 - DNA, 25 bp, Artificial Sequence
  NTC oligonucleotide
caccgtccggagcttctccagtcaa

SEQ ID NO: 6 - DNA, 25 bp, Artificial Sequence
  NTC oligonucleotide
caccgtgcaaagttcagggtaatgg

SEQ ID NO: 7 - DNA, 25 bp, Artificial Sequence
  AHCY oligonucleotide
caccgtttcctcccgtagccgacat

SEQ ID NO: 8 - DNA, 25 bp, Artificial Sequence
  AHCY oligonucleotide
caccgccaggcagccaggccgatgt

SEQ ID NO: 9 - DNA, 25 bp, Artificial Sequence
  AHCY oligonucleotide
caccgtcccgtagccgacatcggcc

SEQ ID NO: 10 - DNA, 25 bp, Artificial Sequence
  MAT2A oligonucleotide
caccgctggaatgatccttcttgct

SEQ ID NO: 11 - DNA, 25 bp, Artificial Sequence
  MAT2A oligonucleotide
caccgtggaatgatccttcttgctg

SEQ ID NO: 12 - DNA, 25 bp, Artificial Sequence
  MAT2A oligonucleotide
caccgtgctgttgactaccagaaag

SEQ ID NO: 13 - DNA, 25 bp, Artificial Sequence
  EZH2 oligonucleotide
caccgcggaaatcttaaaccaagaa

SEQ ID NO: 14 - DNA, 25 bp, Artificial Sequence
  EZH2 oligonucleotide
caccgaccaagaatggaaacagcga

SEQ ID NO: 15 - DNA, 25 bp, Artificial Sequence
  EZH2 oligonucleotide
caccgacagaagtcaggatgtgcac

SEQ ID NO: 16 - DNA, 20 bp, Artificial Sequence
  SARS-CoV-2 N fw primer
ttacaaacattggccgcaaa

SEQ ID NO: 17 - DNA, 18 bp, Artificial Sequence
  SARS-CoV-2 N rev primer
gcgcgacattccgaagaa

SEQ ID NO: 18 - DNA, 20 bp, Artificial Sequence
  RPLP0 fw primer
ggatctgctgcatctgcttg

SEQ ID NO: 19 - DNA, 20 bp, Artificial Sequence
  RPLP0 rev primer
gcgacctggaagtccaacta

(19) United States
(12) Patent Application Publication
(10) Pub. No.: US 2024/0075017 A1
(43) Pub. Date: Mar. 7, 2024

(54) TREATMENT OF CORONAVIRUS INFECTIONS USING SAM CYCLE INHIBITORS

(71) Applicant: TECHNISCHE UNIVERSITÄT MÜNCHEN, München (DE)

(72) Inventors: Andreas PICHLMAIR, Wessling (DE); Valter BERGANT, München (DE); Vincent GRASS, München (DE)

(21) Appl. No.: 18/280,325

(22) PCT Filed: Mar. 8, 2022
(86) PCT No.: PCT/EP2022/055794

§ 371 Date: Sep. 5, 2023

(30) Foreign Application Priority Data
Mar. 8, 2021 (EP) 21161224.7
Sep. 29, 2021 (EP) 21199700.2

(51) Int. Cl. A61K 31/437; A61K 31/136; A61K 31/4745; A61K 31/573; A61K 31/706; A61P 31/14

(52) U.S. Cl. CPC A61K 31/437; A61K 31/136; A61K 31/4745; A61K 31/573; A61K 31/706; A61P 31/14

(57) ABSTRACT

The invention refers to an inhibitor of at least one S-adenosylmethionine (SAM) cycle enzyme for use in preventing or treating coronavirus disease 2019 (COVID-19) in a subject, or for use in preventing or treating of infection with severe acute respiratory syndrome coronavirus-2 (SARS-CoV-2) in a subject, wherein the at least one SAM cycle enzyme is selected from the group consisting of methionine adenosyltransferase, betaine-homocysteine methyltransferase, methionine synthase, methionine synthase reductase and S-adenosylhomocys.

### FIELD OF THE INVENTION

The invention refers to an inhibitor of at least one S-adenosylmethionine (SAM) cycle enzyme for use in preventing or treating coronavirus disease 2019 (COVID-19) in a subject, or for use in preventing or treating infection with severe acute respiratory syndrome coronavirus-2 (SARS-CoV-2) in a subject, wherein the at least one SAM cycle enzyme is selected from the group consisting of methionine adenosyltransferases, betaine-homocysteine methyltransferase, methionine synthase, methionine synthase reductase and S-adenosylhomocysteine hydrolase. Corresponding pharmaceutical compositions are also contemplated.

### BACKGROUND OF THE INVENTION

Viral infections are one of the leading causes of disease burden in human population. Recent pandemics, such as 2003 SARS, 2009 influenza A, 2016 Zika and the 2019/20 SARS-CoV-2, highlighted the requirement for rapid development of antiviral treatments orthogonal to vaccines. As observed in the SARS-CoV-2 pandemic, even developed nations can suffer massively on health, political and socioeconomic levels until the development of a suitable vaccine. Although vaccines recently become available, it remains a challenge to reach herd-immunity and resume pre-pandemic public life while maintaining RO (the basic reproductive rate) below 1, which would correspond to a reduction of COVID-19 cases over time. Moreover, fading immunity in the elderly population, which is at highest risk, antigenic variation of the virus, as well as potential antibody-dependent enhancement of disease can prevent such efforts altogether. Patients that recovered from COVID-19 often suffer from long-term issues associated to lung fibrosis¹, kidney failure² and coagulation disorder³. Small molecule treatment options applied early after infection and that target the virus as well as tissue repair, would be urgently needed to control the pandemics and long-term effects associated with tissue damage.

### OBJECTIVES AND SUMMARY OF THE INVENTION

The inventors discovered a novel class of compounds exhibiting an antiviral effect against SARS-CoV-2. This novel class of compounds is thus useful for preventing and treating COVID-19 and SARS-CoV-2 infection.

The invention is based on the surprising finding that inhibitors of SAM-cycle enzymes of the viral host exhibit an antiviral effect against SARS-CoV-2. The inventors could show that the representative inhibitors of the SAM-cycle enzymes S-adenosylhomocysteine hydrolase (i.e. DZNep and DER), homocysteine methyltransferase (i.e. CBHcy) and methionine adenosyltransferase(s) (i.e. PF-9366, FIDAS-5, MAT2A inhibitor 1) reduced viral growth of SARS-CoV-2 in human lung-derived cells.

Hence, a first aspect of the invention relates to an inhibitor of at least one S-adenosylmethionine (SAM) cycle enzyme for use in preventing or treating coronavirus infectious disease 2019 (COVID-19) in a subject, or for use in preventing or treating SARS-CoV-2 infection in a subject, wherein the at least one SAM cycle enzyme is selected from the group consisting of methionine adenosyltransferase (MAT1A, MAT2A and MAT2B), betaine-homocysteine methyltransferase and betaine-homocysteine methyltransferase 2 (BHMT, BHMT2), methionine synthase (MTR), methionine synthase reductase (MTRR) and S-adenosylhomocysteine hydrolase (AHCY, AHCYL1, AHCYL2).

In one embodiment, the inhibition of at least one of the SAM-cycle enzymes methionine adenosyltransferase (MAT1A, MAT2A and MAT2B), betaine-homocysteine methyltransferase (BHMT, BHMT2), methionine synthase (MTR), methionine synthase reductase (MTRR) and S-adenosylhomocysteine hydrolase (AHCY, AHCYL1, AHCYL2) leads to the decrease of the SAM levels and/or increase of SAH levels and/or decrease of SAM/SAH ratio. As shown exemplary for the AHCY inhibitors DZNep and DER, the MAT1A and MAT2A inhibitor PF-9366, the BHMT/BHMT2 inhibitor CBHcy and the MAT2A inhibitors FIDAS-5 and MAT2A inhibitor 1, the inhibition of SAM-cycle enzymes decreasing the SAM levels and/or increase of SAH levels and/or decrease of SAM/SAH ratio leads to an antiviral effect against SARS-CoV-2.

Thus, in a specific embodiment the at least one SAM cycle enzyme is selected from the group consisting of methionine adenosyltransferase and S-adenosylhomocysteine hydrolase.

The methionine adenosyltransferase may be methionine adenosyltransferase 1A (MAT1A) and/or methionine adenosyltransferase 2A (MAT2A) and/or associated factor without enzymatic activity methionine adenosyltransferase 2B (MAT2B). Preferably, the methionine adenosyltransferase is MAT2A.

The S-adenosylhomocysteine hydrolase may be S-adenosylhomocysteine hydrolase (AHCY), S-adenosylhomocysteine hydrolase like 1 (AHCYL1) or S-adenosylhomocysteine hydrolase like 2 (AHCYL2).

In a specific embodiment, the AHCY inhibitor is selected from the group of analogues of SAM-cycle metabolites SAM, SAH, methionine, homocysteine, adenine or adenosine that cause reduction of SAM levels and/or increase of SAH levels and/or decrease of SAM/SAH ratio under physiological or pathological condition in vitro and/or in vivo, preferably from the structural class of carbocyclic nucleoside analogues or N9-alkylated adenine analogues.

The carbocyclic nucleoside analogues may be DZNep, neplanocin A, 3-deazaaristeromycin and aristeromycin. Preferably, the carbocyclic nucleoside analogue is DZNep.

The N9-alkylated adenine may be DER, 3-deaza-DER, C3-OMeDER and C3-NMeDER and DHPA. Preferably, the N9-alkylated adenine is DER.

Most preferably the AHCY inhibitor is DZNep. The inventors could show by in vivo data that DZNep reduces SARS-CoV-2 viral load and RNA transcripts in lung tissue isolates.

In one embodiment the inhibitor inhibiting methionine adenosyltransferase is a fluorinated N,N-dialkylaminostilbene. Most preferably, the inhibitor is (E)-4-(2-chloro-6-fluorostyryl)-N-methylaniline (FIDAS-5).

In some embodiments, the inhibitor inhibiting methionine adenosyltransferase is selected from the group consisting of FIDAS-5, MAT2A inhibitor 1 and PF-9366.

In some embodiments, the inhibitor further inhibits also enhancer of zeste homolog 2 (EZH2), for example the inhibitor inhibits AHCY and EZH2.

In one embodiment, the at least one SAM cycle enzyme is betaine-homocysteine methyltransferase. In another embodiment, the inhibitor inhibiting betaine-homocysteine methyltransferase is S-(4-Carboxybutyl)-D,L-homocysteine (CBHcy).

In some embodiments, the subject suffers from fibrosis, e.g. lung fibrosis.

In some embodiments, preventing or treating COVID-19 comprises preventing or treating lung fibrosis caused by COVID-19.

In some embodiments, treating or preventing COVID-19 comprises preventing or treating at least one of the symptoms selected from the group consisting of lung fibrosis, interstitial pneumonia, acute lung injury (ALI) and acute respiratory distress syndrome (ARDS).

In one embodiment, the subject is an immune deficient patient, preferably a patient suffering from type I interferon (IFN) deficiency.

In some embodiments, a combination of at least two inhibitors inhibiting two different SAM cycle enzymes is administered (e.g. DZNep and FIDAS-5 are administered in combination).

In some embodiments, the inhibitor is administered in combination with a further therapeutic ingredient.

Another aspect of the invention refers to a pharmaceutical composition comprising the inhibitor together with a pharmaceutically acceptable carrier and an optionally further therapeutic ingredient for use in preventing or treating COVID-19 in a subject, or for use in preventing or treating SARS-CoV-2 infection in a subject.

The further therapeutic ingredient may be selected from the group consisting of protease inhibitors, nucleotide analogues, inhibitors of autophagy, AKT kinase inhibitor, corticosteroids or interferons.

Preferably, the protease inhibitor is a serine protease inhibitor such as camostat or broad spectrum matrix metalloprotease (MMP) inhibitor, such as hydroxamate based inhibitors including BB94, marimastat, prionomastat.

In some embodiments, the further therapeutic ingredient is selected from the group consisting BB94, marimastat, prinomastat, remdesivir, hydroxychloroquine, ipatasertib, dexamethasone and type I interferon, or a combination thereof. The further therapeutic ingredient may be selected from the group consisting of remdesivir and dexamethasone, more preferably remdesivir.

### FIGURE LEGENDS

FIG. 1: Inhibition of SARS-CoV-2-GFP reporter virus growth by 3-deazaneplanocin A (DZNep) in vitro. SARS-CoV-2-GFP viral reporter signal normalised to cell confluence as a measure of virus growth over time (left) or at 48 hours post infection (centre) and confluence of cells as a measure of compound's cytotoxicity (right) upon 6 h pre-treatment of A549-ACE2 cells with indicated concentrations of DZNep, and infection with SARS-CoV-2-GFP virus at MOI 3, as measured by IncuCyte S3 Live-Cell Analysis System. Panels depict data from 4 technical replicates and their mean+/−standard deviation. Vehicle is phosphate buffered saline, h.p.i.—hours post infection.

FIG. 2A: Inhibition of SARS-CoV-2-GFP reporter virus growth by D-eritadenine (DER) in vitro. SARS-CoV-2-GFP viral reporter signal normalised to cell confluence as a measure of virus growth (left) and confluence of cells as a measure of compound's cytotoxicity (right) upon 6 h pre-treatment of A549-ACE2 cells with indicated concentrations of DER, and infection with SARS-CoV-2-GFP virus at MOI 3, as measured by IncuCyte S3 Live-Cell Analysis System. Panels depict data from 4 technical replicates and their mean+/−standard deviation. PBS—phosphate buffered saline.

FIG. 2B: Inhibition of SARS-CoV-2-GFP reporter virus growth by Tazemetostat in vitro. A549-ACE2 cells were pre-treated for 6 h with indicated concentrations of Tazemetostat and infected with SARS-CoV-2-GFP at MOI 3. Normalised integrated GFP intensity is plotted over time (left) and at 48 hours post-infection (right) as a measure of reporter virus growth. Mean of 4 technical replicates (left) and mean+/−sd (right) are depicted. Statistics were calculated using Student's two-sided t-test between indicated treatment concentrations and vehicle control (DMSO, v.). * p<0.05, ** p<0.01, *** p<0.001, h.p.i.—hours post infection.

FIG. 2C: Inhibition of SARS-CoV-2-GFP reporter virus growth by CBHcy in vitro. SARS-CoV-2-GFP viral reporter signal normalised to cell confluence as a measure of virus growth over time (left) or at 48 hours post infection (centre) and confluence of cells as a measure of compound's cytotoxicity (right, cell growth) upon 6 h pre-treatment of A549-ACE2 cells with indicated concentrations of CBHcy, and infection with SARS-CoV-2-GFP virus at MOI 3, as measured by IncuCyte S3 Live-Cell Analysis System. Panels depict data from 8 technical replicates and their mean+/−standard deviation. Statistics were calculated using Student's two-sided t-test between indicated treatment concentrations and vehicle control (DMSO, v.). * p<0.05, ** p<0.01, *** p<0.001, ns—not significant, h.p.i.—hours post infection.

FIG. 3A: Inhibition of SARS-CoV-2 virus growth by FIDAS-5 (fluorinated N,N-dialkylaminostilbene 5) in vitro. SARS-CoV-2-GFP viral reporter signal normalised to cell confluence as a measure of virus growth (top) and confluence of cells as a measure of compound's cytotoxicity (bottom) upon 6 h pre-treatment of A549-ACE2 cells with indicated concentrations of FIDAS-5, and infection with SARS-CoV-2-GFP virus at MOI 3, as measured by IncuCyte S3 Live-Cell Analysis System. Panel depicts 6 technical replicates and their mean+/−standard deviation, and is representative of 3 independent repeats. Two-sided Student t-test was used for p-value calculation comparing virus growth at distinct treatment conditions with vehicle-treated control as indicated. ns—not significant (p>0.05), * p<0.05, ** p<0.01, *** p<0.001.

FIG. 3B: Inhibition of SARS-CoV-2 virus growth by FIDAS-5 and FIDAS-5/DZNep co-treatment in vitr. SARS-CoV-2-GFP viral reporter signal normalised to cell confluence as a measure of virus growth (top) and confluence of cells as a measure of compound's cytotoxicity (bottom) upon 6 h pre-treatment of A549-ACE2 cells with indicated concentrations of FIDAS-5 and vehicle or DZNep, and infection with SARS-CoV-2-GFP virus at MOI 3, as measured by IncuCyte S3 Live-Cell Analysis System. Panel depicts 4 technical replicates and their mean+/−standard deviation. Two-sided Student t-test was used for p-value calculation comparing virus growth at distinct treatment conditions with vehicle-treated control as indicated. ns—not significant (p>0.05), * p<0.05, ** p<0.01, *** p<0.001, v. vehicle (FIDAS-5: DMSO, DZNep: PBS).

FIG. 3C: Inhibition of SARS-CoV-2 virus growth by DZNep, FIDAS-5 and CBHcy in Vero E6 cells in vitro. Vero-E6 cells were pre-treated for 6 h with indicated concentrations of DZNep, FIDAS-5 and CBHcy, and infected with SARS-CoV-2 at MOI 0.01. 48 hours post-infection, produced infectious progeny was tittered on Vero E6 cells and expressed as log 10 plaque forming units per unit volume. Plots depict mean+/−sd of 3 technical replicates. v—vehicle.

FIG. 4: Inhibition of SARS-CoV-2-GFP reporter virus growth by MAT2A inhibitor 1 (MI1, A) and PF-9366 (B) in vitro. SARS-CoV-2-GFP viral reporter signal normalised to cell confluence as a measure of virus growth over time (left) or at 48 hours post infection (centre) and confluence of cells as a measure of compound's cytotoxicity (right) upon 6 h pre-treatment of A549-ACE2 cells with indicated concentrations of MI1 or PF-9366, and infection with SARS-CoV-2-GFP virus at MOI 3, as measured by IncuCyte S3 Live-Cell Analysis System. Panel depicts 4 technical replicates and their mean+/−standard deviation. Two-sided Student t-test was used for p-value calculation comparing virus growth at distinct treatment conditions with vehicle-treated control as indicated. ns—not significant (p>0.05), * p<0.05, ** p<0.01, *** p<0.001, v.—vehicle (DMSO), h.p.i.—hours post infection.

FIG. 5: Schematic representation of the proposed mode of action for the activity of 3-deazaneplanocin A in preventing or treating SARS-CoV-2 infection, COVID-19 and underlying or virus-caused fibrosis in a subject. 3-Deazaneplanocin A (DZNep) is a known inhibitor of S-adenosylhomocysteine hydrolase (AHCY, EC 3.3.1.1) and Enhancer of zeste homolog 2 (EZH2, direct inhibition), a host SAM-dependent methyltransferase (MTase). Inhibition of AHCY by DZNep is known to cause the increase of SAH amount and the reduction of SAM to SAH ratio, both known biomarkers of cellular methylation capacity, which in turn inhibit activity of host and viral SAM-dependent MTases including EZH2 (indirect inhibition). Direct and indirect inhibition of EZH2, together with inhibition of host and viral SAM-dependent MTases, reduces SARS-CoV-2 virus proliferation and allows prevention and treatment of SARS-CoV-2 infection, COVID-19 and underlying or COVID-19-caused fibrosis in a subject.

FIG. 6: Schematic representation of the proposed mode of action for the activity of SAM-cycle component inhibitors in preventing or treating SARS-CoV-2 infection, COVID-19 and underlying fibrotic conditions or fibrotic conditions caused by COVID-19 in a subject. Inhibition of SAM-cycle components is known to reduce SAM amount and/or increase SAH amount and/or reduce SAM to SAH ratio. SAM and SAH amounts and SAM/SAH ratio are known biomarkers of cellular methylation capacity (SAM and SAM/SAH correlate with, and SAH inversely correlates with cellular methylation capacity). Reduced cellular methylation capacity inhibits activity of host and viral SAM-dependent methyltransferases (MTases), reducing SARS-CoV-2 virus proliferation. Reduction of cellular methylation capacity inhibits activity of host and viral SAM-dependent MTases including enhancer of zeste homolog 2 (EZH2) that is known to be involved in tissue repair and regeneration, which synergizes with reduction of SARS-CoV-2 virus proliferation and allows prevention and treatment of underlying or COVID-19-caused fibrosis in a subject.

FIG. 7: Inhibition of SARS-CoV-2 virus growth by 3-deazaneplanocin A (DZNep) in vitro. A549-ACE2 cells were pre-treated with indicated concentrations of DZNep for 6 h and either mock-infected or infected with SARS-CoV-2 (strain MUC-IMB-1) at MOI 3. 24 hours post-infection, a western blot analysis was performed with immunostaining against SARS-CoV-2 nucleoprotein (NP) as a measure of virus growth, and human beta actin (ACTB) as a loading control. v.—vehicle control (phosphate buffered saline).

FIG. 8: Inhibition of SARS-CoV-2-MUC-IMB-1 or SARS-CoV-2-B.1.1.7 (Alpha variant) or B.1.617.2 (Delta variant) by DZNep in vitro. A549-ACE2 cells were pre-treated with indicated concentrations of DZNep and infected with indicated variants of SARS-CoV-2 at MOI 3. 24-hours post-infection, virus load was quantified using RT-qPCR against SARS-CoV-2 N using host-encoded RPLP0 for input normalisation. Two-sided Student t-test was used for p-value calculation comparing conditions as indicated. v.—vehicle control (phosphate buffered saline).

FIG. 9: Inhibition of SARS-CoV-2 and lack of inhibition of SARS-CoV virus growth by 3-deazaneplanocin A (DZNep) in vitro. A549-ACE2 cells were pre-treated with indicated concentrations of DZNep or vehicle (v., phosphate buffered saline) for 6 h and infected with SARS-CoV-2 (strain MUC-IMB-1) or SARS-CoV (strain Frankfurt 1) at MOI 3. 24 hours post-infection, a western blot analysis was performed with immunostaining against SARS-CoV-2 and SARS-CoV nucleoprotein (NP) as a measure of virus growth, and human beta actin (ACTB) as a loading control. NP band intensity, normalised to ACTB band intensity, is shown as percentage of virus-matched vehicle-treated control. v.—vehicle control (phosphate buffered saline).

FIG. 10: Inhibition of SARS-CoV-2 and lack of inhibition of SARS-CoV virus growth by 3-deazaneplanocin A (DZNep) in primary human lung cells ex vivo. Normal human bronchial cells (NHBE) were pre-treated with indicated concentrations of DZNep for 6 h and infected with SARS-CoV-2 virus (strain MUC-IMB-1) or SARS-CoV virus (strain Frankfurt 1) at MOI 3. 24 hours post-infection, an immunofluorescent staining was performed against SARS-CoV-2 and SARS-CoV nucleoprotein (NP). Normalised integrated intensity of NP immunostaining as a measure of virus growth is depicted as SARS-CoV NP signal and SARS-CoV-2 NP signal, and is shown as percentage of vehicle-treated virus- and donor-matched control. Cell confluence as a measure of compound's cytotoxicity is shown as percentage of vehicle-treated virus- and donor-matched control. Panel depicts mean of between 2 and 4 technical replicates+/−standard deviation from 3 donors. Two-sided donor-wise paired Student t-test was used for p-value calculation comparing control-normalised NP signal at distinct DZNep concentrations with vehicle-treated virus-matched controls as indicated.

FIG. 11: Inhibition of SARS-CoV-2 virus growth by 3-deazaneplanocin A (DZNep) in vitro, using non-targeting control (NTC) or STAT1 KO A549-ACE2 cells pre-treated with vehicle (phosphate buffered saline, PBS) or interferon alpha (IFNα). SARS-CoV-2-GFP viral reporter signal area normalised to cell confluence as a measure of virus growth upon 6 h pre-treatment of A549-ACE2 NTC or STAT1 KO cells with indicated concentrations of interferon alpha (IFNα) and/or DZNep, and infection with SARS-CoV-2-GFP virus at MOI 3, as measured by IncuCyte S3 Live-Cell Analysis System. Panel depicts mean of 2 technical replicates and is representative of 3 independent repeats.

FIG. 12: Inhibition of SARS-CoV-2 virus growth by 3-deazaneplanocin A (DZNep) in vrq, using non-targeting control (NTC), AHCY and MAT2A KO A549-ACE2 cells pre-treated with vehicle (phosphate buffered saline, PBS) or DZNep. SARS-CoV-2-GFP viral reporter signal area normalised to cell confluence as a measure of virus growth upon 6 h pre-treatment of A549-ACE2 NTC-, AHCY- and MAT2A-KO cells with indicated concentration of DZNep or vehicle (phosphate buffered saline), and infection with SARS-CoV-2-GFP virus at MOI 3, as measured by IncuCyte S3 Live-Cell Analysis System. Panel depicts 3 technical replicates with mean+/−standard deviation. Two-sided Student t-test was used for p-value calculation comparing virus growth at distinct times post-infection with treatment-matched NTC control. * p<0.05, vehicle—phosphate buffered saline.

FIG. 13A: Inhibition of SARS-CoV-2 and lack of inhibition of SARS-CoV virus growth by 3-deazaneplanocin A (DZNep) in vitro. Heatmap depicting log 2 LFQ intensities of SARS-CoV-2 (indicated with suffix_SARS2) and SARS-CoV (indicated with suffix_SARS) proteins as measured in full proteome analysis of A549-ACE2 cells, pre-treated for 6 h with 0.75 μM 3-deazaneplanocin A (DZNep) and infected with SARS-CoV-2 (strain MUC-IMB-1) or SARS-CoV (strain Frankfurt 1) at MOI 3 for 24 h.

FIG. 13B: Proteins significantly regulated by 3-deazaneplanocin A (DZNep) in NHBEs infected with SARS-CoV or SARS-CoV2 in vitro. Proteins, differentially expressed upon DZNep treatment of NHBEs in the contexts of SARS-CoV and SARS-CoV-2 infections were used for network diffusion analysis in order to identify genes, functionally interacting with them. The graph shows a cluster of genes found significant in this analysis related to fibrosis and coagulation, and inflammation. The network is overlaid with LASSO-based log 2 fold change between SARS-CoV-2 infected DZNep and vehicle treated NHBEs.

FIG. 13C: Proteins significantly regulated by 3-deazaneplanocin A (DZNep) in NHBEs infected with SARS-CoV or SARS-CoV2 in vitro. Heatmap depicting log 2 fold changes of host protein abundances between indicated conditions as measured in full proteome analysis of NHBE cells, pre-treated for 6 h with 0.75 μM 3-deazaneplanocin A (DZNep) and infected with SARS-CoV-2 (strain MUC-IMB-1) or SARS-CoV (strain Frankfurt 1) at MOI 3 for 36 h. Changes in protein abundances were analysed according to depicted scheme using LASSO-based linear model followed by fixed LASSO inference based p-value estimation as described in the methods section. Upper panel shows upregulation, bottom panel shows downregulation of proteins.

FIG. 13D: Inhibition of SARS-CoV-2 and lack of inhibition of SARS-CoV virus growth by 3-deazaneplanocin A (DZNep) in vitro. Mass spectrometry based analysis of cells treated with DZNep and infected with SARS-CoV-2 and SARS-CoV. A549-ACE2s and NHBEs were pre-treated for 6 h with 0.75 and 1.5 μM DZNep, respectively, or vehicle, and infected with SARS-CoV-2 or SARS-CoV at MOI 3 for 24 h (A549-ACE2) or 36 h (NHBEs). Donor-normalised LFQ abundances of viral nucleoprotein (N) and spike (S) in indicated conditions are depicted. Statistics were calculated using Student's two-sided t-test as indicated.

FIG. 14: 3-deazaneplanocin A (DZNep) treatment induces cytoprotective and tissue-preserving response and cell-intrinsic antiviral response in vitro. A subnetwork of significant subset of reactome FI (v2019) functional interaction network, as determined by network diffusion analysis of proteins, significantly regulated by 3-deazaneplanocin A (DZNep). Proteins, significantly up- and down-regulated by 6 h 0.75 μM DZNep pre-treatment of A549-ACE2 cells, either mock-infected or infected with SARS-CoV-2 (strain MUC-IMB-1) or SARS-CoV (strain Frankfurt 1), are highlighted with round or square thick borders, respectively. Portions of subnetwork, containing proteins involved in cytoprotective and tissue-preserving responses or cell-intrinsic antiviral responses are shown.

FIG. 15: Inhibition of SARS-CoV-2-GFP reporter virus growth by broad-spectrum matrix metalloprotease (MMP) inhibitors from hydroxamate family with optional co-treatment with DZNep in vitro. SARS-CoV-2-GFP viral reporter signal normalised to cell confluence as a measure of virus growth (top) and confluence of cells as a measure of compound's cytotoxicity (bottom) upon 6 h pre-treatment of A549-ACE2 cells with indicated concentrations of inhibitors and vehicle or DZNep, and infection with SARS-CoV-2-GFP virus at MOI 3, as measured by IncuCyte S3 Live-Cell Analysis System. Panel depicts 4 technical replicates and their mean+/−standard deviation. Two-sided Student t-test was used for p-value calculation comparing conditions as indicated. ns p>0.05, * p<0.05, ** p<0.01, *** p<0.001, v. vehicle (MMP inhibitors: DMSO, DZNep: PBS).

FIG. 16: Inhibition of SARS-CoV-2-GFP reporter virus growth by Remdesivir with optional co-treatment with DZNep in vitro. SARS-CoV-2-GFP viral reporter signal normalised to cell confluence as a measure of virus growth (top) and confluence of cells as a measure of compound's cytotoxicity (bottom) upon 6 h pre-treatment of A549-ACE2 cells with indicated concentrations of Remdesivir and vehicle or DZNep, and infection with SARS-CoV-2-GFP virus at MOI 3, as measured by IncuCyte S3 Live-Cell Analysis System. Panel depicts 4 technical replicates and their mean+/−standard deviation. Two-sided Student t-test was used for p-value calculation comparing conditions as indicated. ns p>0.05, * p<0.05, ** p<0.01, *** p<0.001, v. vehicle.

FIG. 17: Inhibition of SARS-CoV-2-GFP reporter virus growth by hydroxychloroquine with optional co-treatment with DZNep in vitro. SARS-CoV-2-GFP viral reporter signal normalised to cell confluence as a measure of virus growth (top) and confluence of cells as a measure of compound's cytotoxicity (bottom) upon 6 h pre-treatment of A549-ACE2 cells with indicated concentrations of hydroxychloroquine and vehicle or DZNep, and infection with SARS-CoV-2-GFP virus at MOI 3, as measured by IncuCyte S3 Live-Cell Analysis System. Panel depicts 4 technical replicates and their mean+/−standard deviation. Two-sided Student t-test was used for p-value calculation comparing conditions as indicated. ns p>0.05, * p<0.05, ** p<0.01, *** p<0.001, v. vehicle.

FIG. 18: Inhibition of SARS-CoV-2-GFP reporter virus growth by Ipatasertib with optional co-treatment with DZNep in vitro. SARS-CoV-2-GFP viral reporter signal normalised to cell confluence as a measure of virus growth (top) and confluence of cells as a measure of compound's cytotoxicity (bottom) upon 6 h pre-treatment of A549-ACE2 cells with indicated concentrations of Ipatasertib and vehicle or DZNep, and infection with SARS-CoV-2-GFP virus at MOI 3, as measured by IncuCyte S3 Live-Cell Analysis System. Panel depicts 4 technical replicates and their mean+/−standard deviation. Two-sided Student t-test was used for p-value calculation comparing conditions as indicated. ns p>0.05, * p<0.05, ** p<0.01, *** p<0.001, v. vehicle.

FIG. 19: Inhibition of SARS-CoV-2-GFP reporter virus growth by dexamethasone with optional co-treatment with DZNep in vitro. SARS-CoV-2-GFP viral reporter signal normalised to cell confluence as a measure of virus growth (top) and confluence of cells as a measure of compound's cytotoxicity (bottom) upon 6 h pre-treatment of A549-ACE2 cells with indicated concentrations of dexamethasone and vehicle or DZNep, and infection with SARS-CoV-2-GFP virus at MOI 3, as measured by IncuCyte S3 Live-Cell Analysis System. Panel depicts 4 technical replicates and their mean+/−standard deviation. Two-sided Student t-test was used for p-value calculation comparing conditions as indicated. ns p>0.05, * p<0.05, ** p<0.01, *** p<0.001, v. vehicle.

FIG. 20: Inhibition of SARS-CoV-2-GFP reporter virus growth by DZNep and co-treatment with indicated concentrations of Dexamethasone or vehicle (DMSO) in vitro. SARS-CoV-2-GFP viral reporter signal normalised to cell confluence as a measure of virus growth (left) and confluence of cells as a measure of compound's cytotoxicity (right) upon 6 h pre-treatment of A549-ACE2 cells with indicated concentrations treatments, and infection with SARS-CoV-2-GFP virus at MOI 3, as measured by IncuCyte S3 Live-Cell Analysis System at 48 h post-infection. Panel depicts 4 technical replicates and their mean+/−standard deviation. Two-sided Student t-test was used for p-value calculation comparing individual conditions with DZNep vehicle control. ns p>0.05, * p<0.05, ** p<0.01, *** p<0.001. v. vehicle (phosphate buffered saline).

FIG. 21: Inhibition of SARS-CoV-2-GFP reporter virus growth by DZNep and co-treatment with indicated concentrations of Remdesivir or vehicle (DMSO) in vitro. SARS-CoV-2-GFP viral reporter signal normalised to cell confluence as a measure of virus growth (left) and confluence of cells as a measure of compound's cytotoxicity (right) upon 6 h pre-treatment of A549-ACE2 cells with indicated concentrations treatments, and infection with SARS-CoV-2-GFP virus at MOI 3, as measured by IncuCyte S3 Live-Cell Analysis System at 48 h post-infection. Panel depicts 4 technical replicates and their mean+/−standard deviation. Two-sided Student t-test was used for p-value calculation comparing individual conditions with DZNep vehicle control. ns p>0.05, * p<0.05, ** p<0.01, *** p<0.001, v. vehicle (phosphate buffered saline).

FIG. 22: Schematic representation of the SAM-cycle. SAM-cycle component is selected from the group consisting of S-Adenosylhomocysteine hydrolases (AHCY/AHCYL1/AHCYL2) and methionine adenosyltransferases (MAT1A, MAT2A and MAT2B) and methionine synthases (BHMT, BHMT2, MTR and MTRR). Names, targets and structures of representative SAM-cycle inhibitors are shown (DER—D-eritadenine, DZNep—3-deazaneplanocin A, FIDAS-5—fluorinated N,N-dialkylaminostilbene 5, MI1—MAT2A inhibitor 1, PF-9366-2-(7-Chloro-5-phenyl-[1,2,4]triazolo[4,3-a]quinolin-1-yl)-N,N-dimethylethan-1-amine) and CBHcy (S-(4-Carboxybutyl)-D,L-homocysteine; 5-(3-Amino-3-carboxypropyl)sulfanyl-pentanoic acid).

FIG. 23: Schematic representation of the biomarkers of cellular methylation capacity, and how they can be influenced to induce reduction of cellular methylation capacity. Reduction of SAM levels, increase of SAH levels and reduction of SAM/SAH ratio cause a reduction of cellular methylation capacity, leading to inhibition of host and pathogen SAM-dependent methyltransferases.

FIG. 24: Schematic representation of the proposed mode of action for the activity of SAM-cycle component inhibitors in preventing or treating SARS-CoV-2 infection and COVID-19 in a subject. SAM-cycle component inhibitors, where SAM-cycle component is selected from the group consisting of S-Adenosylhomocysteine Hydrolase (AHCY, AHCYL1 and AHCYL2, collectively termed AHCYs), Methionine Adenosyltransferases (collectively abbreviated MAT: MAT1A, MAT2A and MAT2B) and methionine synthases (BHMT, BHMT2, MTR and MTRR, collectively termed MSs), exemplified by representative AHCY inhibitors 3-deazaneplanocin A (DZNep) and D-eritadenine (DER), MAT1A/MAT2A/MAT2B inhibitor PF-9366, MAT2A inhibitors FIDAS-5 and MAT2A inhibitor 1 (MI1) and MS inhibitor CBHcy. Inhibition of these SAM-cycle components is known to reduce SAM amount and/or increase SAH amount and/or reduce SAM to SAH ratio. SAM and SAH amounts and SAM/SAH ratio are known biomarkers of cellular methylation capacity (SAM and SAM/SAH correlate with, and SAH inversely correlates with cellular methylation capacity). Reduction of cellular methylation capacity inhibits activity of host and viral SAM-dependent methyltransferases, reducing SARS-CoV-2 virus proliferation and allows prevention and treatment of SARS-CoV-2 infection and COVID-19 in a subject.

FIG. 25: IL-6 and IP10-production by NHBEs treated with mock-, SARS-CoV or SARS-CoV-2 with optional pre-treatment with DZNep or its vehicle in vitro. NHBE cells from 5 (IP10 measurements, SARS-CoV-2+/−DZNep) or 6 (all other conditions) donors were pre-treated for 6 h with 3-deazaneplanocin A (0.75 μM) or vehicle (PBS) and infected with SARS-CoV-2-MUC-IMB-1 or SARS-CoV-Frankfurt-1 at MOI 3. Cell supernatant was harvested 24 h post-infection and analysed for human IL6 (top) and IP10 (bottom) by ELISA. Two-sided Student t-test was used for p-value calculation on log-transformed values between indicated conditions before donor-wise normalisation to vehicle treated mock controls.

FIG. 26: C57BL/6 mice infected with SARS-CoV-2 and treated with DZNep in vivo. Mice were infected with 250 pfu of SARS-CoV-2 B.1.351 intranasal and treated at day zero and day one with 10 μg DZNep intranasal or vehicle control. Lungs of infected animals were isolated 48 h after infection.

FIG. 26A: Viral load was quantified by titration of lung homogenate supernatant (n=5 per condition) on Vero E6 cells. The graph shows mean+/−standard deviation of the lung titer, expressed as log 10 infectious viral particles per unit mass of lungs.

FIG. 26B: Viral transcripts for SARS-CoV-2 nucleoprotein (N), membrane protein (M) and envelope protein (L) were quantified in RNA, isolated from lungs of infected animals (n=8 per condition), by RT-qPCR. The graph shows negative ΔCt values, as normalised to 185 rRNA (M, E) or Actb transcript, and respective mean+/−standard deviation. Statistics were calculated using Student's two-sided t-test as indicated.

FIG. 27: Synergism of DZNep with IFN-α and Remdesivir.

FIG. 27A: A549-ACE2 cells were pretreated for 6 h with indicated concentrations of IFN-α and DZNep and infected with SARS-CoV-2-GFP at MOI 1. Means of normalized integrated GFP intensities of 6 independently infected wells are shown as a measure of the reporter virus growth at 24 h post-infection alongside the combination index (CI)⁷⁴ as a measure of treatments' synergy. The data demonstrates strong synergism of a combination of DZNep and INF-α in vitro. FIG. 27B: A549-nRFP-ACE2 cells were pretreated for 6 h with indicated concentrations of Remdesivir and DZNep and infected with SARS-CoV-2-GFP at MOI 1. Means of normalized integrated GFP intensities of 5 independently infected wells are shown as a measure of the reporter virus growth at 24 h post-infection alongside the combination index (CI)⁷⁴ as a measure of treatments' synergy. The data demonstrates a high degree of synergy of a combination of DTNep and Remdesivir in vitro. Presented data is representative of 3 independent repeats.

### DETAILED DESCRIPTION OF THE INVENTION

Before the invention is described in detail with respect to some of its preferred embodiments, the following general definitions are provided.

The present invention as illustratively described in the following may suitably be practiced in the absence of any element or elements, limitation or limitations, not specifically disclosed herein.

The present invention will be described with respect to particular embodiments and with reference to certain figures but the invention is not limited thereto but only by the claims.

Where the term ‘comprising’ is used in the present description and claims, it does not exclude other elements. For the purposes of the present invention, the term “consisting of” is considered to be a preferred embodiment of the term “comprising of”. If hereinafter a group is defined to comprise at least a certain number of embodiments, this is also to be understood to disclose a group which preferably consists only of these embodiments.

For the purposes of the present invention, the term “obtained” is considered to be a preferred embodiment of the term “obtainable”. If hereinafter e.g. an antibody is defined to be obtainable from a specific source, this is also to be understood to disclose an antibody which is obtained from this source.

Where an indefinite or definite article is used when referring to a singular noun, e.g. “a”, “an” or “the”, this includes a plural of that noun unless something else is specifically stated. The terms “about” or “approximately” in the context of the present invention denote an interval of accuracy that the person skilled in the art will understand to still ensure the technical effect of the feature in question. The term typically indicates deviation from the indicated numerical value of ±10%, and preferably of ±5%.

Technical terms are used by their common sense. If a specific meaning is conveyed to certain terms, definitions of terms will be given in the following in the context of which the terms are used.

The invention is based on the surprising finding that inhibitors of SAM-cycle enzymes of the viral host, i.e. cells of a human subject, exhibit an antiviral effect against SARS-CoV-2. The present data supports the rationale that the inhibition of human SAM-cycle enzymes lowering the cellular methylation capacity, i.e. the ratio of SAM to SAH and/or lowering of SAM concentration and/or increasing the SAH concentration, leads to an antiviral effect against SARS-CoV-2. The inventors concluded that decrease of the SAM/SAH ratio and/or decrease of SAM concentration and/or increase of SAH concentration lead not only to the inhibition of the host but also to the inhibition of the viral MTases of SARS-CoV-2. The inventors could show that the representative inhibitors of the SAM-cycle enzyme AHCY (i.e. DZNep and DER), MAT1A and MAT2A (i.e. PF-9366), BHMT/BHMT2 (i.e. CBHcy) and MAT2A (i.e. FIDAS-5 and MAT2A inhibitor 1) reduced viral growth of SARS-CoV-2 in human lung cells. Both inhibition of AHCY and inhibition of MAT2A alone or inhibition of MAT1A and MAT2A leads to the reduction of the cellular methylation capacity. Without being bound to any theory, decreasing the cellular methylation capacity in turn leads to a broad-spectrum inhibition of host and viral MTases finally provoking the antiviral efficacy of these inhibitors. In particular, the inventors discovered that DZNep is an inhibitor having a particular strong antiviral effect against SARS-CoV-2.

Accordingly, a first aspect of the invention relates to an inhibitor of an S-adenosylmethionine (SAM) cycle enzyme for use in preventing or treating coronavirus disease 2019 (COVID-19) in a subject, or for use in preventing or treating of SARS-CoV-2 in a subject, wherein the at least one SAM cycle enzyme is selected from the group consisting of methionine adenosyltransferase, betaine-homocysteine methyltransferase, methionine synthase, methionine synthase reductase and S-adenosylhomocysteine hydrolase.

SAM-cycle is a metabolic circuit in human cells that produces main methyl group donor S-adenosylmethionine (SAM) and recycles the product-inhibitor of methylation reactions S-adenosylhomocysteine (SAH). Enzymes, involved in production of SAM from methionine, are methionine adenosyltransferases MAT1A, MAT2A and their modulator without enzymatic activity MAT2B, of which MAT2A is widely accepted to play a dominant role in most tissues including lungs. The only enzyme currently known to metabolise SAH to homocysteine is adenosylhomocysteine hydrolase AHCY. Homocysteine is metabolized back to methionine to complete the cycle by distinct enzymes, betaine homocysteine methyltransferase (BHMT, BHMT2) and methionine synthase (MTR). For its optimal activity, MTR requires an additional enzyme methionine synthase reductase (MTRR).

The term SAM cycle enzyme also includes associated factors of the SAM cycle enzymes.

Methionine adenosyltransferase (EC 2.5.1.6) catalyzes the synthesis of S-adenosylmethionine. The methionine adenosyltransferase may be methionine adenosyltransferase 1A (MAT1A), also termed S-adenosylmethionine synthase isoform type-1, and/or methionine adenosyltransferase 2A (MAT2A), also termed S-adenosylmethionine synthase isoform type-2, and/or their modulator without enzymatic activity methionine adenosyltransferase 2B (MAT2B). Preferably the methionine adenosyltransferase is MAT2A. It is known that inhibition of MAT2A leads to a decrease in SAM concentration without concomitant change in SAH concentration⁴, and the skilled person can reasonably conclude that inhibition of MAT1A and/or MAT2B has the same effect.

Betaine-homocysteine methyltransferase (BHMT, BHMT2) also termed betaine-homocysteine S-methyltransferase (EC 2.1.1.5) is a zinc metallo-enzyme. It catalyzes the transfer of a methyl group from trimethylglycine and a hydrogen ion from homocysteine to produce dimethylglycine and methionine. It is known that inhibition of betaine-homocysteine methyltransferase leads to a reduction of SAM levels, increase in SAH levels and decrease in SAM/SAH ratio⁵.

Methionine synthase (MTR) (EC 2.1.1.13) is an enzyme that requires vitamin B12 (cobalamin) and catalyses the reaction between (6S)-5-methyl-5,6,7,8-tetrahydrofolate and L-homocysteine forming (6S)-5,6,7,8-tetrahidrofolate and L-methionine. Methionine synthase required a separate protein, methionine synthase reductase (MTRR) (EC 1.15.1.18), to retain its activity. Methionine synthase reductase is required to maintain activity of the catalytic site of methionine synthase, and in its absence, methionine synthase rapidly loses its enzymatic activity. Since MTR/MTRR catalyze a reaction that produces the same product (methionine) as BHMT and BHMT2, the skilled person can reasonably conclude that inhibition of MTR and/or MTRR would affect SAM and SAH levels in the same manner as inhibition of BHMT and/or BHMT2.

S-adenosylhomocysteine hydrolase also termed adenosylhomocysteinase (3.3.1.1) hydrolyses S-adenosyl-L-homocysteine into adenosine and homocysteine. The term includes for example AHCY, AHCYL1 and AHCYL2. Preferably, the S-adenosylhomocysteine hydrolase is AHCY. It is known that AHCY inhibitors cause an increase in SAH concentration⁶ and decrease SAM/SAH ratio⁷, that reduced expression of AHCY causes the increase in SAH concentration and the decrease of SAM/SAH ratio⁸ and that in subjects with AHCY deficiency, serum SAM/SAH ratio is reduced⁹.

In a specific embodiment, the at least one SAM cycle enzyme is selected from the group consisting of methionine adenosyltransferase and S-adenosylhomocysteine hydrolase. In a preferred embodiment the at least one SAM cycle enzyme is AHCY or MAT2A. In an even more preferred embodiment the at least one SAM cycle enzyme is AHCY.

Inhibition of at least one SAM cycle enzyme selected from methionine adenosyltransferase, betaine-homocysteine methyltransferase, methionine synthase, methionine synthase reductase and S-adenosylhomocysteine hydrolase, leads to the decrease of the ratio of SAM/SAH and/or the decrease of SAM concentration and/or the increase in SAH concentration. By increasing the amount of SAH and/or decreasing the amount of SAM and/or reducing the ratio of SAM/SAH, cellular methylation capacity is reduced, and its decrease leads to an inhibition of SAM-dependent methyltransferases (MTases) regardless of the organism of origin. Thus, not only the host MTases but also pathogen MTases are inhibited or at least decreased in their activity. The inventors conclude that the decrease of SAM concentration and/or the increase of SAH concentration and/or decrease of SAM/SAH ratio and subsequent broad-spectrum inhibition of MTases is the main molecular cause of antiviral efficacy of DZNep against SARS-CoV-2. Hence, inhibition of SAM cycle enzymes decreasing the cellular methylation capacity also inhibits the MTases of host and SARS-CoV-2, as exemplified by the AHCY inhibitor DER and the MAT1A and MAT2A inhibitor PF-9366, the BHMT/BHMT2 inhibitor CBHcy and MAT2A inhibitors FIDAS-5 and MAT2A inhibitor 1.

The skilled person is aware of methods to measure SAM and SAH levels, as for example described in provided references^{7,10}.

SARS-CoV-2, also termed 2019-nCoV, refers to severe acute respiratory syndrome coronavirus-2 firstly described by Zhu et al., 2019¹¹ and variants thereof, e.g. without limitation variant B.1.1.7 (also known as 20I/501Y.V1, VOC 202012/01), B.1.351 (20H/501Y.V2) and P1, as defined by the Coronaviridae Study Group of the International Committee on Taxonomy of Viruses (ICTV-CSG)¹². Accordingly, SARS-CoV-2 is used to denote all variants of a virus, according to ICTV belonging to realm Riboviria, kingdom Orthornavirae, phylum Pisuviricota, class Pisoniviricetes, order Nidovirales, family Coronaviridae, genus Betacoronavirus, subgenus Sarbecovirus, species Severe acute respiratory syndrome-related coronavirus, strain Severe acute respiratory syndrome coronavirus 2 (SARS-CoV-2).

The term “inhibitor” includes small molecules, antibodies and binding fragments thereof, non-antibody protein scaffold proteins, aptamers and nucleotide based molecules, such as siRNAs or gRNAs. In preferred embodiments, the inhibitor is a small molecule.

The skilled person understand that the term ‘inhibiting’ also includes significantly reducing.

The AHCY inhibitor may be selected from the group of analogues of SAM-cycle metabolites SAM, SAH, methionine, homocysteine, adenine or adenosine that cause reduction of SAM levels and/or increase of SAH levels and/or decrease of SAM/SAH ratio under physiological or pathological condition in vitro and/or in vivo, preferably from the structural class of carbocyclic nucleoside analogues or N9-alkylated adenine analogues. In a specific embodiment, the carbocyclic nucleoside analogues may be DZNep, neplanocin A, 3-deazaaristeromycin and aristeromycin. Preferably, the carbocyclic nucleoside analogue is DZNep. The N9-alkylated adenine may be DER, 3-deaza-DER, C3-OMeDER and C3-NMeDER and DHPA. Preferably, the N9-alkylated adenine is DER.

The identification of AHCY inhibitors and further examples for adenosylhomocysteine hydrolase (EC 3.3.1.1) inhibitors were previously described^{10,13-37}. The skilled person is aware of methods for testing whether an enzyme is an inhibitor of AHCY, which were previously described^{6,10} and are also commercially available. In a preferred embodiment, the inhibitors of AHCY include DZNep and D-eritadenine (DER).

Surprisingly, the inventors found that 3-Deazaneplanocin A, also termed DZNep, C-c3Ado, exhibits an antiviral activity for SARS-CoV-2.

Thus, in particular the invention relates to DZNep for use in preventing or treating coronavirus disease 2019 (COVID-19) in a subject, or for use in preventing or treating SARS-CoV-2 infection in a subject. Surprisingly, only very low doses of DZNep are necessary to achieve a high antiviral activity against SARS-CoV.2.

More particularly, the invention relates to DZNep or a pharmaceutical acceptable salt thereof for use in treating coronavirus disease 2019 (COVID-19) in a subject, and/or for use in treating SARS-CoV-2 infection in a subject. DZNep as used herein also refers to encapsulated or packaged versions of DZNep. In a particular embodiment, DZNep is liposome-packaged DZNep In one embodiment, treatment with DZNep or a pharmaceutically acceptable salt thereof leads to a reduction of SARS-CoV-2 viral transcripts compared to untreated control subjects. In particular, treatment with DZNep or pharmaceutically acceptable salts thereof leads to a reduction of SARS-CoV-2 viral transcripts compared to untreated control subjects in vivo, preferably in the respiratory system of the subjects. Accordingly, one embodiment is directed to DZNep or pharmaceutical acceptable salts thereof for use in reducing SARS-CoV-2 viral transcripts in the respiratory system of a subject infected with SARS-CoV-2.

In one embodiment, treatment with DZNep or a pharmaceutically acceptable salt thereof inhibits virus replication in the respiratory system of a subject.

Accordingly, one embodiment is directed to DZNep or pharmaceutical acceptable salts thereof for use in inhibiting virus replication in the respiratory system of a subject infected with SARS-CoV-2.

In one embodiment, the respiratory system comprises one or more of nose, nasal cavities, sinuses, pharynx, larynx, trachea, bronchi, bronchiole, alveolar ducts and alveoli. In one embodiment, the respiratory system is lungs.

Another embodiment is directed to DZNep or pharmaceutical acceptable salts thereof for use in treating lung fibrosis, interstitial pneumonia, acute lung injury (ALI), acute respiratory distress syndrome (ARDS), kidney injury, such as proteinuria and acute kidney injury, and vasculopathy and other extrapulmonary manifestations of COVID-19 (e.g. thrombotic complications, myocardial dysfunction and arrhythmia, acute coronary syndromes, gastrointestinal symptoms, hepatocellular injury, hyperglycemia and ketosis, neurologic illnesses, ocular symptoms, and dermatologic complications) as previously described³⁸.

In one embodiment, treatment with DZNep or pharmaceutically acceptable salts thereof during SARS-CoV-2 infection leads to a reduction of one or more lung fibrosis biomarkers, preferably wherein the lung fibrosis biomarkers are selected from COL4A1 (Collagen alpha-1(IV) chain), MMP14 (Matrix metalloproteinase-14) and SERPINE1 (serine protease inhibitor E1).

In yet another embodiment, treatment with DZNep or pharmaceutically acceptable salts thereof during SARS-CoV-2 infection leads to an up-regulation of one or more factors counteracting fibrotic processes, preferably selected from ELAFIN/PI3 (elastase-specific protease inhibitor/peptidase inhibitor 3), SLPI (secretory leukocyte protease inhibitor) and ECM1 (Extracellular matrix protein 1).

In yet another embodiment, treatment with DZNep or pharmaceutically acceptable salts thereof during SARS-CoV-2 infection leads to a reduction of factors of extrinsic coagulation cascade, preferably selected from F3 (coagulation factor 111) and TFPI2 (tissue factor pathway inhibitor 2) and/or reduction of factors of plasminogen activation system, preferably selected from PAI1 (plasminogen activator inhibitor-1), PLAT (tissue plasminogen activator) and PLAU (plasminogen activator, urokinase type).

In yet another embodiment, treatment with DZNep or pharmaceutically acceptable salts thereof during SARS-CoV-2 infection leads to changes in the abundance of innate immunity related factors, preferably selected from IL1RN (interleukin-1 receptor antagonist), C3 (complement component 3) and TNFAIP3/A20 (Tumor necrosis factor, alpha-induced protein 3).

SARS-CoV-2 infection leads to an increase in IL-6 (interleukin-6) secretion and a repression of type-I interferon signalling. In one embodiment, treatment with DZNep or pharmaceutically acceptable salts thereof during SARS-CoV-2 infection leads to a reduction in IL-6 expression. In another embodiment, treatment with DZNep or pharmaceutically acceptable salts thereof leads to an increase of interferon secretion, preferably IP-10 (interferon gamma-induced protein 10) secretion.

In some embodiments, the subject suffers from fibrosis, e.g. lung fibrosis. In some embodiments, preventing or treating COVID-19 comprises preventing or treating lung fibrosis caused by COVID-19.

In some embodiments, the subject suffers from coagulopathy. In some embodiments, preventing or treating COVID-19 comprises preventing or treating coagulopathy caused by COVID-19.

The skilled person is aware of methods for identification of inhibitors of SAM cycle enzymes and testing of inhibitors of SAM cycle enzymes.

Inhibitors of methionine adenosyltransferase include analogues of SAM-cycle metabolites SAM, SAH, methionine, homocysteine, adenine or adenosine that cause reduction of SAM levels and/or increase of SAH levels and/or decrease of SAM/SAH ratio under physiological or pathological conditions in vitro and/or in vivo. More specifically, inhibitors of MAT1A and/or MAT2A include fluorinated N,N-dialkylaminostilbene agents such as (E)-4-(2-chloro-6-fluorostyryl)-N-methylaniline (FIDAS-5) as well as other compounds structurally related to FIDAS-5, as for example described in^{39,40} and substituted Pyrazolo[1,5-a]pyrimidin-7(4H)-on or derivatives (such as MAT2A inhibitor 1 and other structurally related compounds as for example described in patent application WO 2018/045071 A1). Inhibitors of MAT1A and MAT2A include benzodiazepine analogs where quinolone ring system replaces benzodiazepine ring system (PF-9366)⁴¹. In preferred embodiments the inhibitor of MAT2A is selected from the group consisting of FIDAS-5, MAT2A inhibitor 1 and PF-9366. MAT2A inhibitor 1 is also indicated as MI1 herein. Further examples for methionine adenosyltransferase (EC 2.5.1.6) inhibitors are described in⁴²⁻⁴⁶. The skilled person is aware of methods for testing whether an enzyme is an inhibitor of MAT1A/MAT2A/MAT2B, which are described for example in the literature⁴¹.

Inhibitors of betaine-homocysteine methyltransferase (EC 2.1.1.5) include analogues of SAM-cycle metabolites SAM, SAH, methionine, homocysteine, adenine or adenosine that cause reduction of SAM levels and/or increase of SAH levels and/or decrease of SAM/SAH ratio under physiological or pathological conditions in vitro and/or in vivo. Inhibitors of betaine-homocysteine methyltransferase are for example described in⁴⁷⁻⁶⁰.

Inhibitors of methionine synthase (EC 2.1.1.13) include analogues of SAM-cycle metabolites SAM, SAH, methionine, homocysteine, adenine or adenosine that cause reduction of SAM levels and/or increase of SAH levels and/or decrease of SAM/SAH ratio under physiological or pathological conditions in vitro and/or in vivo. An exemplary inhibitor of methionine synthase, in particular BHMT, is CBHcy (S-(4-Carboxybutyl)-D,L-homocysteine). Further inhibitors of methionine synthase (EC 2.1.1.13) are for example described in^{61,62}.

Inhibitors of methionine synthase reductase include analogues of SAM-cycle metabolites SAM, SAH, methionine, homocysteine, adenine or adenosine that cause reduction of SAM levels and/or increase of SAH levels and/or decrease of SAM/SAH ratio under physiological or pathological conditions in vitro and/or in vivo. Inhibitors of methionine synthase reductase (EC 1.16.1.8) are for example described in⁶³⁻⁶⁶.

The compounds of the present invention may be administered in the form of pharmaceutically acceptable salts.

The term “pharmaceutically acceptable salt” refers to a salt which possesses the effectiveness of the parent compound and which is not biologically or otherwise undesirable (e.g., is neither toxic nor otherwise deleterious to the recipient thereof). Suitable salts include acid addition salts which may, for example, be formed by mixing a solution of the compound of the present invention with a solution of a pharmaceutically acceptable acid such as hydrochloric acid, sulfuric acid, acetic acid, trifluoroacetic acid, or benzoic acid. When the compounds of the invention carry an acidic moiety, suitable pharmaceutically acceptable salts thereof can include alkali metal salts (e.g., sodium or potassium salts), alkaline earth metal salts (e.g., calcium or magnesium salts), and salts formed with suitable organic ligands such as quaternary ammonium salts. Also, in the case of an acid (—COOH) or alcohol group being present, pharmaceutically acceptable esters can be employed to modify the solubility or hydrolysis characteristics of the compound.

The pharmaceutically acceptable salt of DZNep may be for example the hydrochloride salt of DZNep.

Surprisingly, the combination of DZNep and FIDAS-5 showed a synergistic effect. In some cases, the combination of DZNep and FIDAS-5 showed at least an additive effect. Hence, some embodiments relate to the combination of DZNep and FIDAS-5 or pharmaceutical acceptable salts thereof for use in treating COVID-19 in a subject, and/or for use in treating SARS-CoV-2 infection in a subject.

Accordingly, one embodiment of the invention refers to the combination of at least two inhibitors selected from the group consisting of inhibitor of methionine adenosyltransferase, inhibitor of betaine-homocysteine methyltransferase, inhibitor of methionine synthase, inhibitor of methionine synthase reductase and inhibitor of S-adenosylhomocysteine hydrolase, for use in treating COVID-19 in a subject, and/or for use in treating SARS-CoV-2 infection in a subject.

A preferred embodiment refers to the combination of an inhibitor of methionine adenosyltransferase and an inhibitor of S-adenosylhomocysteine hydrolase. A particular preferred embodiment refers to the combination of an inhibitor of MAT2A and an inhibitor of AHCY.

“Combination” as used herein refers to the administration of two or more drugs (i.e. DZNep and FIDAS-5) to the patient either separately or in a mixture containing the two or more drugs (i.e. DZNep and FIDAS-5). In the case of separate administration the drugs can be administered at the same time point or at a different time point.

In some embodiments, the inhibitor is administered in combination with a further therapeutic ingredient.

Another aspect of the invention refers to a pharmaceutical composition comprising the inhibitor according to any one of the preceding claims together with a pharmaceutically acceptable carrier and an optionally further therapeutic ingredient for use in preventing or treating COVID-19 in a subject, or for use in preventing or treating SARS-CoV-2 infection in a subject.

The further therapeutic ingredient may be selected from the group consisting of protease inhibitors, nucleotide analogues, inhibitors of autophagy, AKT kinase inhibitor, corticosteroids or interferons.

The protease inhibitor may be a broad spectrum matrix metalloprotease (MMP) inhibitor, such as of BB94, marimastat, prionomastat or a serine protease inhibitor such as camostat.

In some embodiments the further therapeutic ingredient is selected from the group consisting of BB94, marimastat, prinomastat, remdesivir, hydroxychloroquine, ipatasertib, dexamethasone and type I interferon, or a combination thereof. The further therapeutic ingredient may preferably be selected from the group consisting of remdesivir and dexamethasone, more preferably remdesivir.

COVID-19 is a contagious disease caused by SARS-CoV-2. In particular COVID-19 refers to a disease as defined in the current international classification of diseases (ICD-11, World Health Organisation, Version: 09/2020). More particularly, COVID-19 is used to denote the disease, diagnosed clinically, epidemiologically or otherwise, irrespective of whether laboratory testing is conclusive, inconclusive or not available.

Treating or preventing of COVID-19 may include treating or preventing at least one of lung fibrosis, interstitial pneumonia, acute lung injury (ALI), acute respiratory distress syndrome (ARDS), aveolar damage, kidney injury, vasculopathy, cardiac injury, acute myocardial injury, chronic damage to the cardiovascular system, thrombosis and venous thromboembolism, in a patient with COVID-19. In a specific embodiment lung fibrosis, interstitial pneumonia, acute lung injury (ALI), acute respiratory distress syndrome (ARDS), kidney injury, such as proteinuria and acute kidney injury, and vasculopathy are triggered by COVID-19.

Kidney injury may be without limitation e.g. proteinuria and acute kidney injury.

The subject may be a mammal. Preferably, the subject is a human.

### EXAMPLES

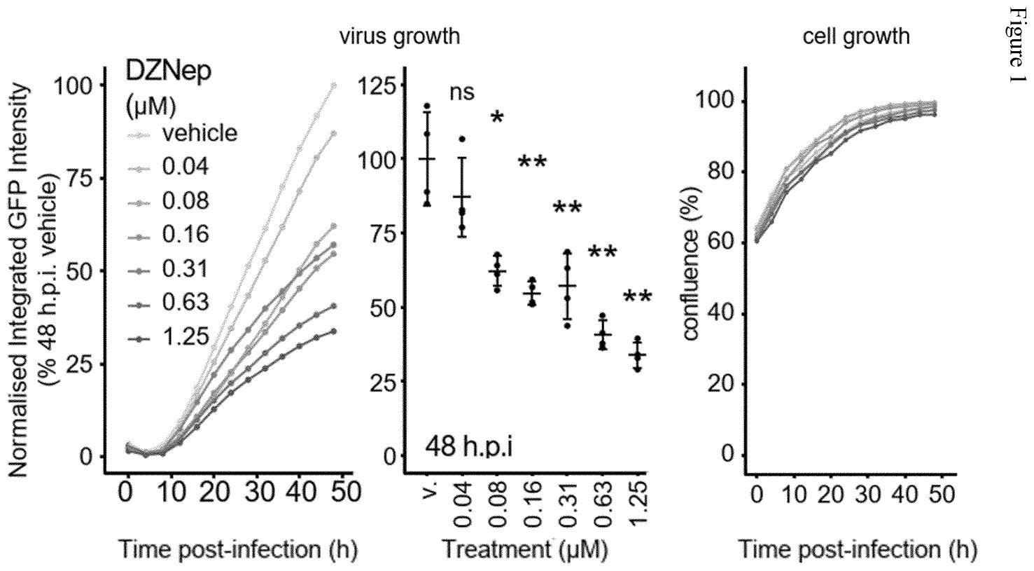

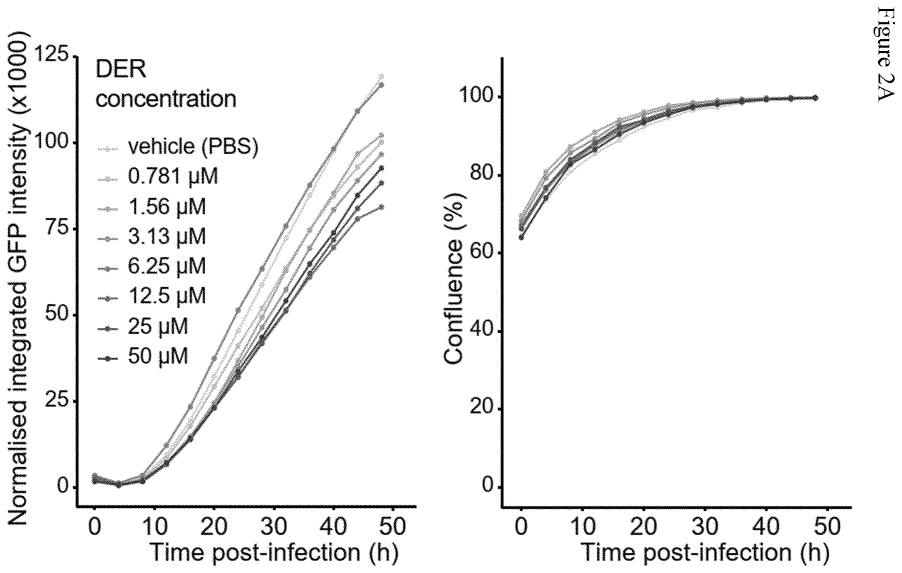

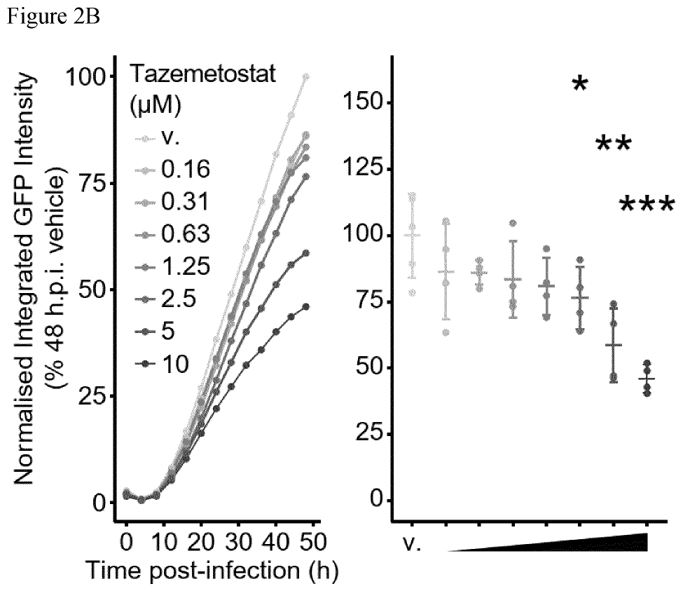

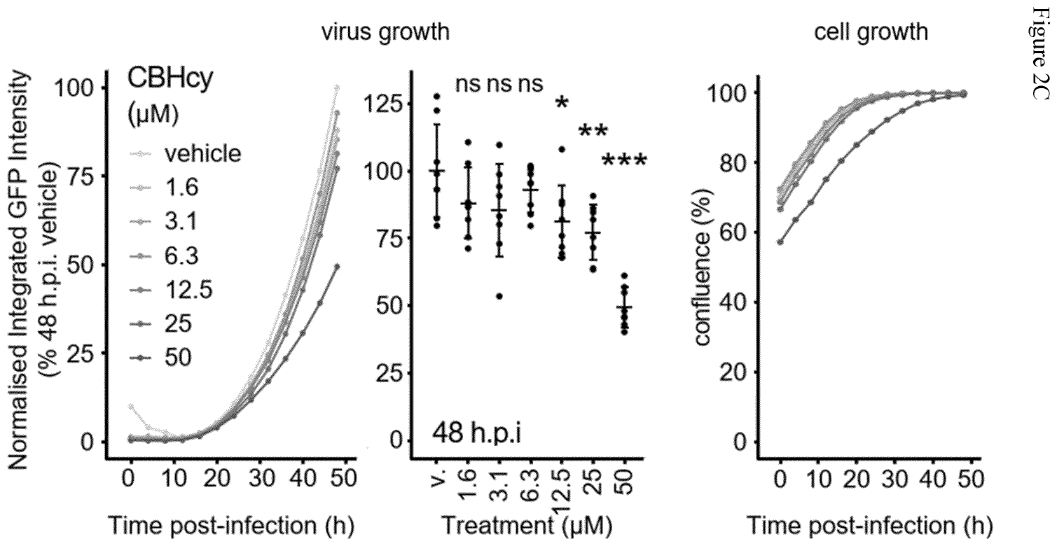

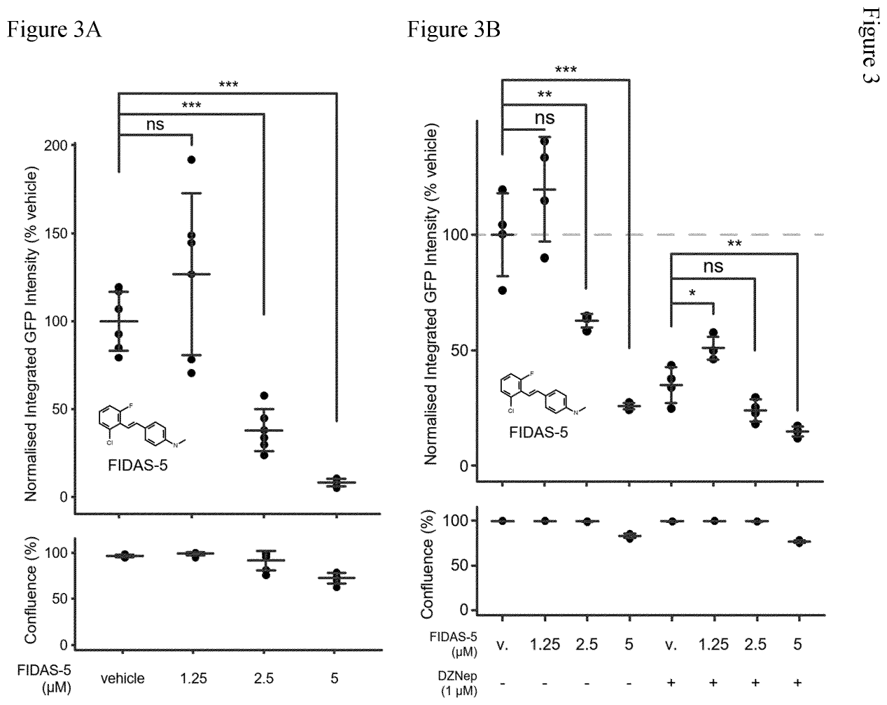

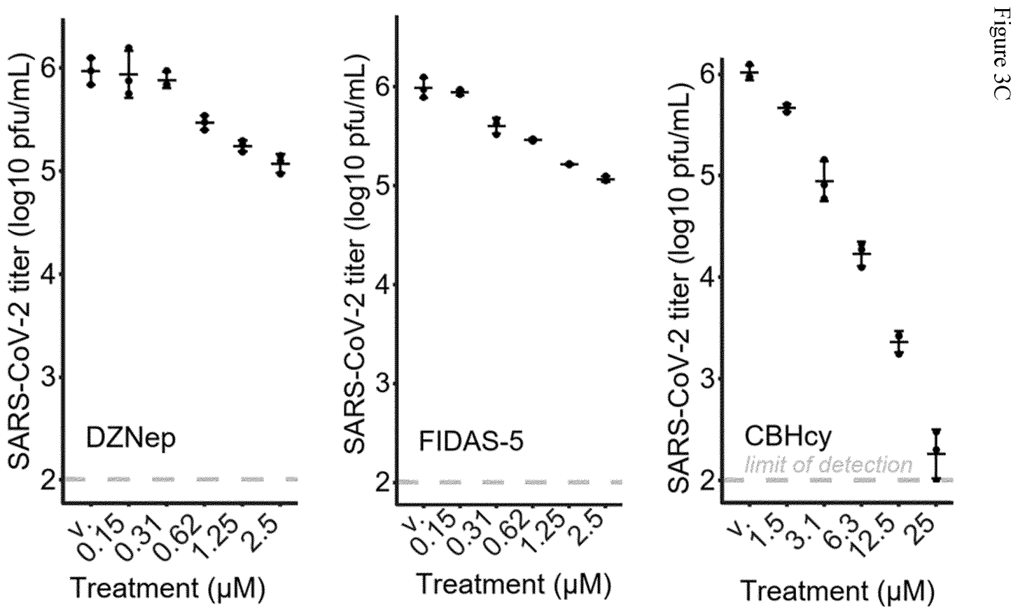

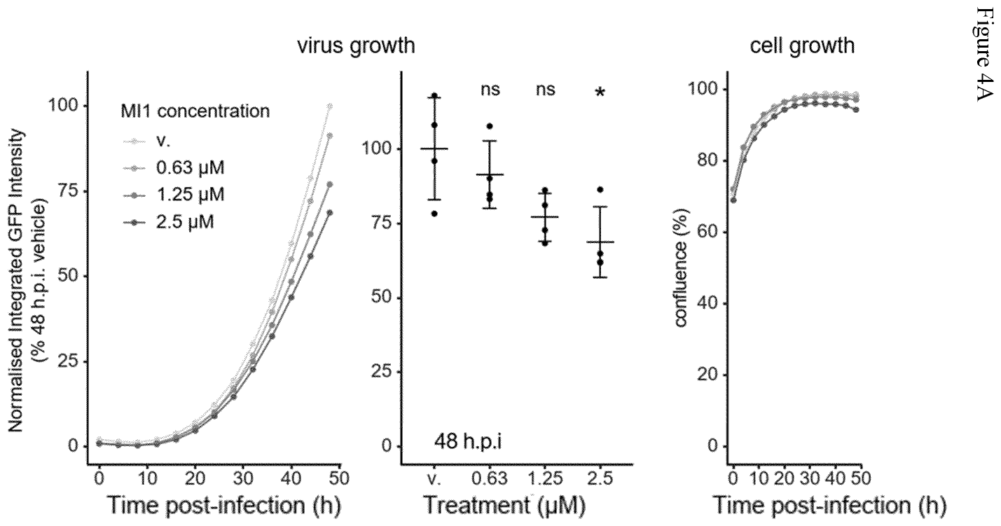

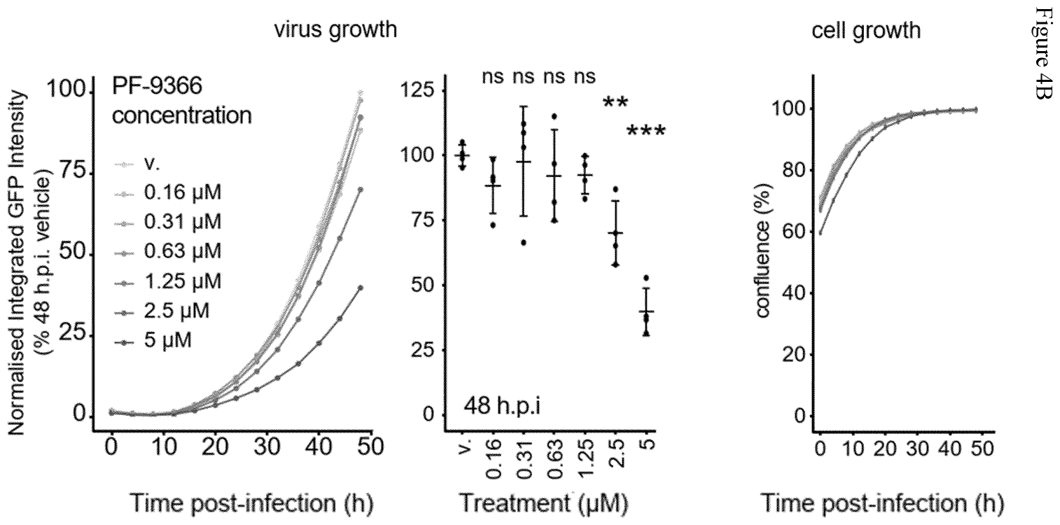

Using real time live-cell fluorescent imaging, we followed growth of SARS-CoV-2-GFP reporter virus⁶⁷ in A549-ACE2 lung-derived cell line⁶⁸ upon 6 h pre-treatment with various inhibitors. We show that representative inhibitors of SAM-cycle, specifically 3-deazaneplanocin A (DZNep, FIG. 1), D-eritadenine (DER, FIG. 2A), S-(4-Carboxybutyl)-D,L-homocysteine (CBHcy, FIG. 2C), (E)-4-(2-chloro-6-fluorostyryl)-N-methylaniline (FIDAS-5, FIG. 3A), MAT2A inhibitor 1 (MI1, FIG. 4A) and 2-(7-Chloro-5-phenyl-[1,2,4]triazolo[4,3-a]quinolin-1-yl)-N,N-dimethylethan-1-amine) (PF-9366, FIG. 4B) have antiviral activity against SARS-CoV-2. Surprisingly, DZNep showed a particularly strong antiviral efficacy (FIG. 1). Co-treatment with AHCY and MAT inhibitors (DZNep and FIDAS-5, respectively), exhibited synergistic antiviral activity (FIG. 3B), indicating that cotreatment with multiple SAM-cycle inhibitors has increased therapeutic potential over treatment with a single inhibitor. We also followed growth of SARS-CoV-2-GFP reporter virus in A549-ACE2 lung-derived cell line upon 6 h pre-treatment with tazemetostat, an inhibitor of EZH2, and observed a treatment-dependent decrease of virus proliferation (FIG. 2B).

Additionally, we evaluated antiviral efficacy of DZNep, FIDAS-5 and CBHcy in Vero E6 cells. Towards this, we pre-treated Vero E6 cells with SAM-cycle inhibitors at a range of concentrations, infected them with wild-type (wt) SARS-CoV-2 and after 48 hours quantified the amount of released viral progeny in the supernatant by plaque assay. In line with the reporter virus assays, we observed dose-dependent suppression of the wt SARS-CoV-2 for the SAM-cycle inhibitors (FIG. 3C).

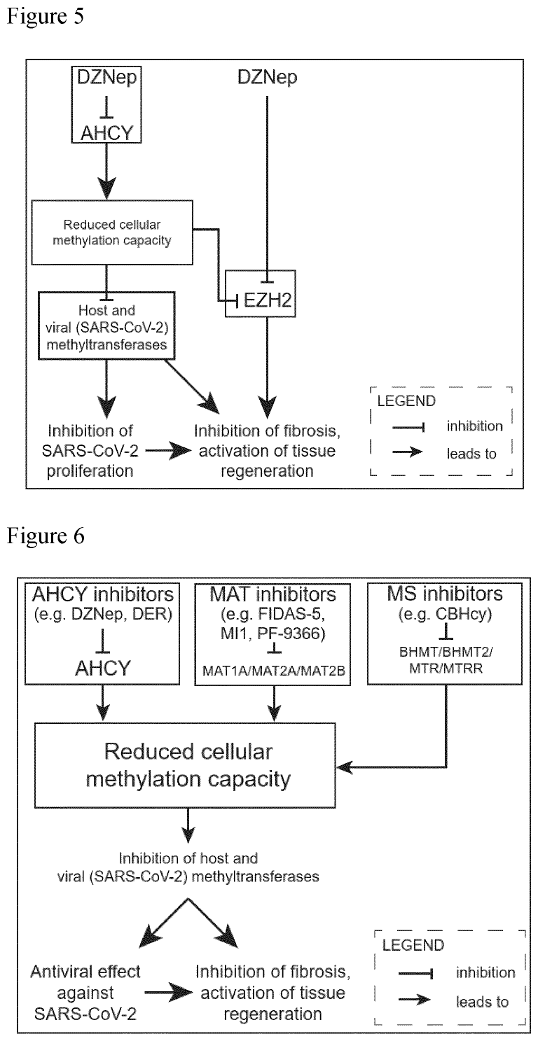

Inhibition of EZH2 by DZNep was previously associated to antifibrotic effect in various organs, including lungs⁶⁹. Inhibition of SARS-CoV-2 virus growth and also inhibition of host MTases such as EZH2, for example directly by DZNep (FIG. 5) or indirectly by other SAM-cycle component inhibitors (FIG. 6), may allow for treatment of pulmonary and extra-pulmonary manifestations of COVID-19 affecting function of various organs, and specifically lung fibrosis.

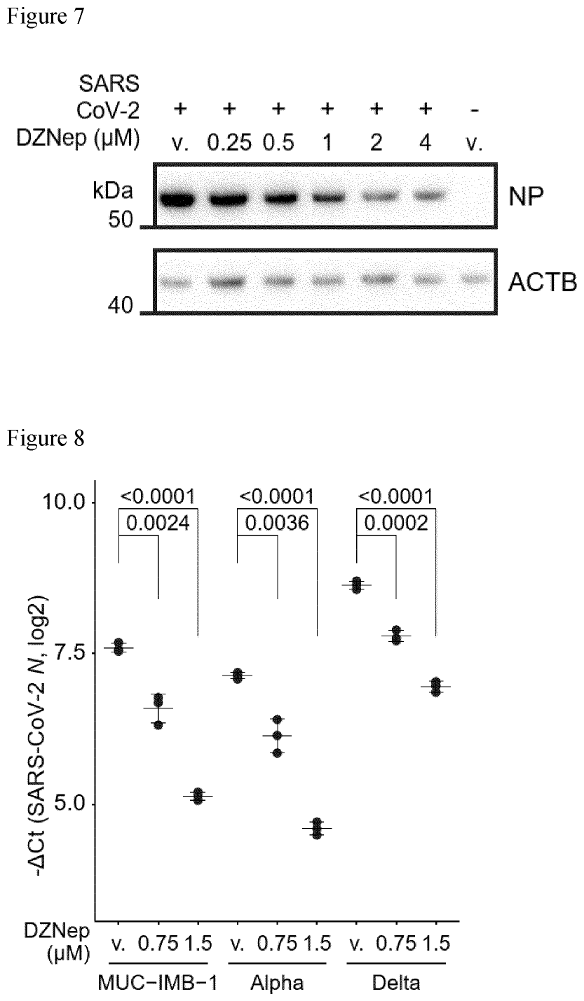

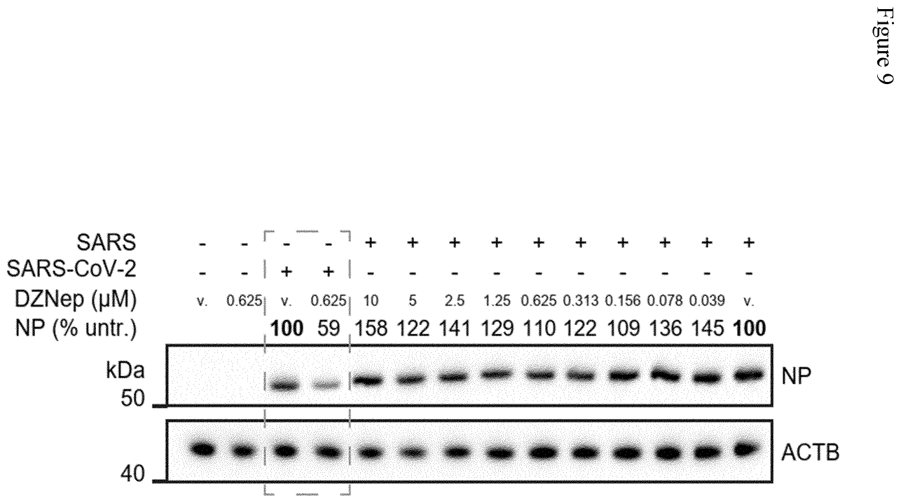

We further validated the antiviral effect of SAM-cycle component inhibitor DZNep by pre-treating A549-ACE2 cells with various concentrations of DZNep, infecting cells with SARS-CoV-2 (variant MUC-IMB-1) and analysing virus nucleoprotein (NP) expression 24 hours post-infection by western blot (FIG. 7). These results further corroborate that SAM-cycle component inhibitors exhibit potent antiviral effect against SARS-CoV-2. In addition, we compared the antiviral efficacy of DZNep against variants MUC-IMB-1 and B.1.1.7 (also known as the UK variant or Alpha variant) and B.1.617.2 (also known as Delta variant) by pre-treating A549-ACE2 cells for 6 h with different concentrations of DZNep, infecting with different variants and analysing viral gene expression 24 hours post-infection by RT-qPCR. We provide supporting evidence that DZNep has similar antiviral activity against the two variants (FIG. 8).

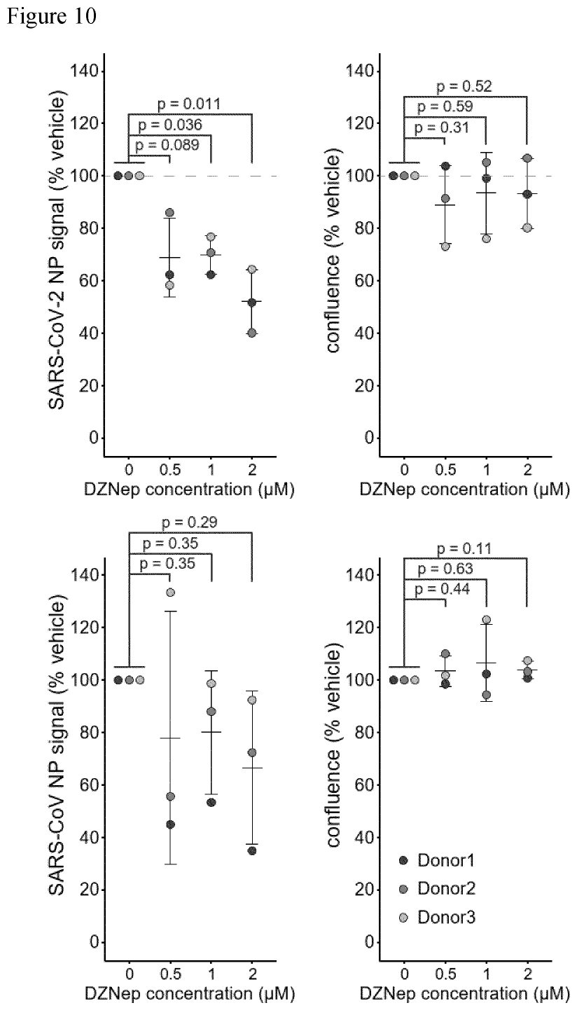

It was described previously that DZNep did not show antiviral activity against SARS-CoV in an in vivo infection model⁷⁰. In line with previous art, we do not observe an antiviral effect of DZNep against the SARS-CoV (variant Frankfurt-1) by probing expression of viral protein N (nucleoprotein) upon 6 h-pretreatment of A549-ACE2 cells and infecting with SARS-CoV (or SARS-CoV-2 as control) for 24 hours (FIG. 9). However, contrary to the teaching of the prior art, we could surprisingly show that DZNep is a potent inhibitor of SARS-CoV-2 in primary human ex-vivo infection model (FIG. 10).

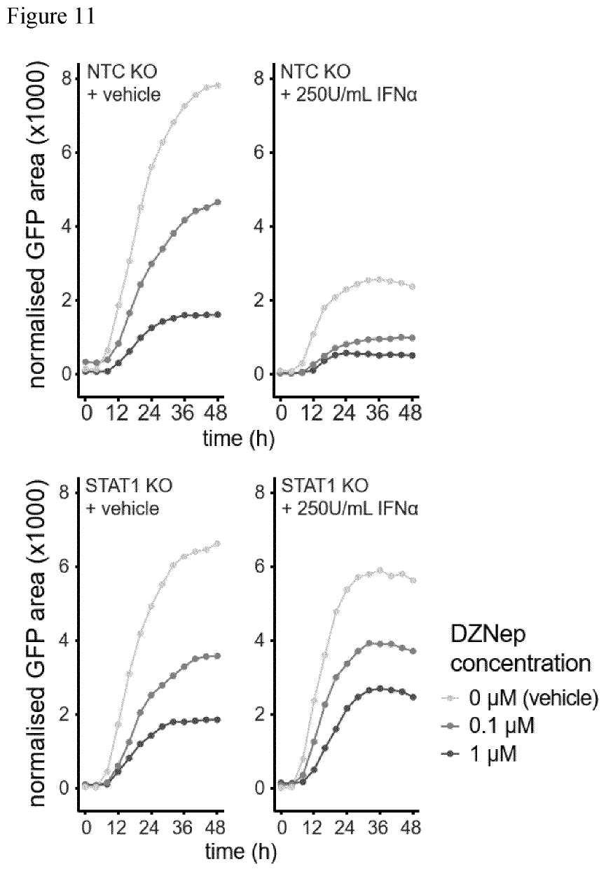

In order to test if type 1 interferon response, one of the hallmark responses of cell intrinsic immunity, is involved in antiviral activity of DZNep against SARS-CoV-2, we generated STAT1 and non-targeting control (NTC) knock-out cell lines. We pre-treated them for 6 h with DZNep and/or interferon alpha and infected them with SARS-CoV-2-GFP reporter virus. By following reporter virus growth using live cell fluorescent imaging we show that cell intrinsic immunity, in particular STAT1 dependent interferon response, does not play a major role in SARS-CoV-2 inhibition in vitro (FIG. 11). Interferon treatment, in particular type 1 interferon treatment, is thus considered a cotreatment option that may synergise with antiviral efficacy of DZNep. Taken together, SAM-cycle component inhibitors represent compounds with a novel mode of antiviral activity against SARS-CoV-2.

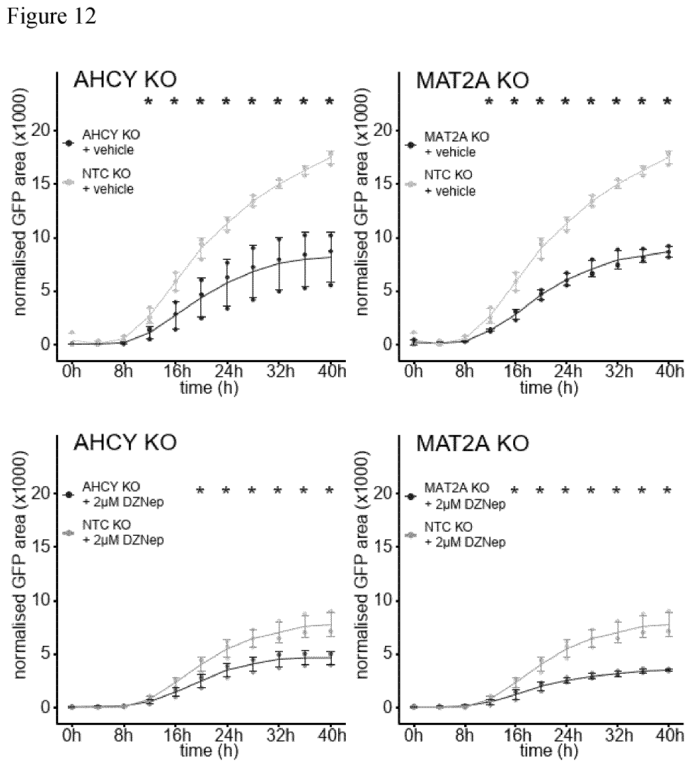

To further corroborate the SAM-cycle components as central in antiviral efficacy of used treatments, we performed a genetic ablation (knock-out, KO) of AHCY and MAT2A. Using this system, orthogonal to pharmaceutical intervention, we show that AHCY and MAT2A KO cells facilitate massively reduced virus growth when compared to control (FIG. 12). This further corroborates that SAM-cycle components are important host factors for SARS-CoV-2 and that their inhibition leads to a potent repression of the virus.

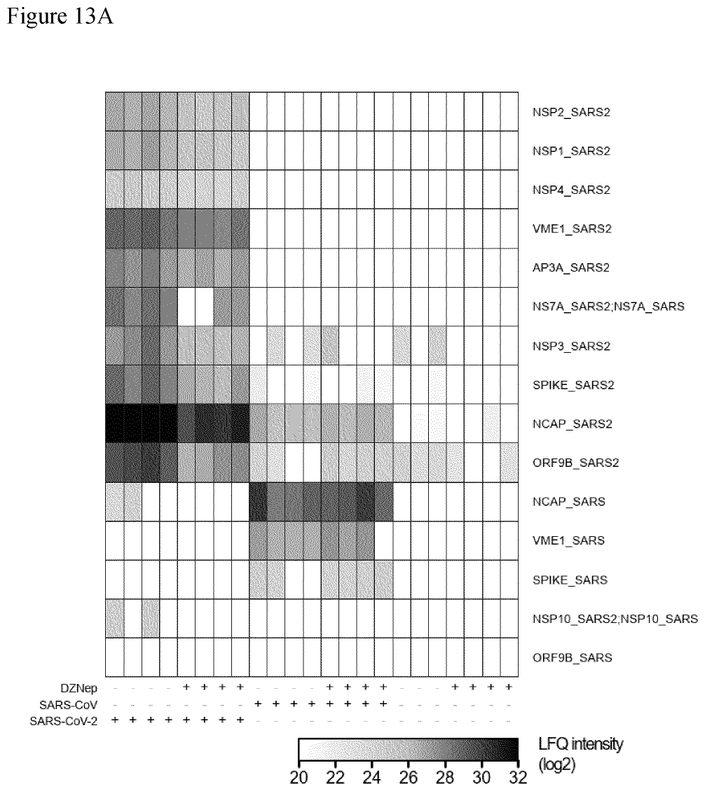

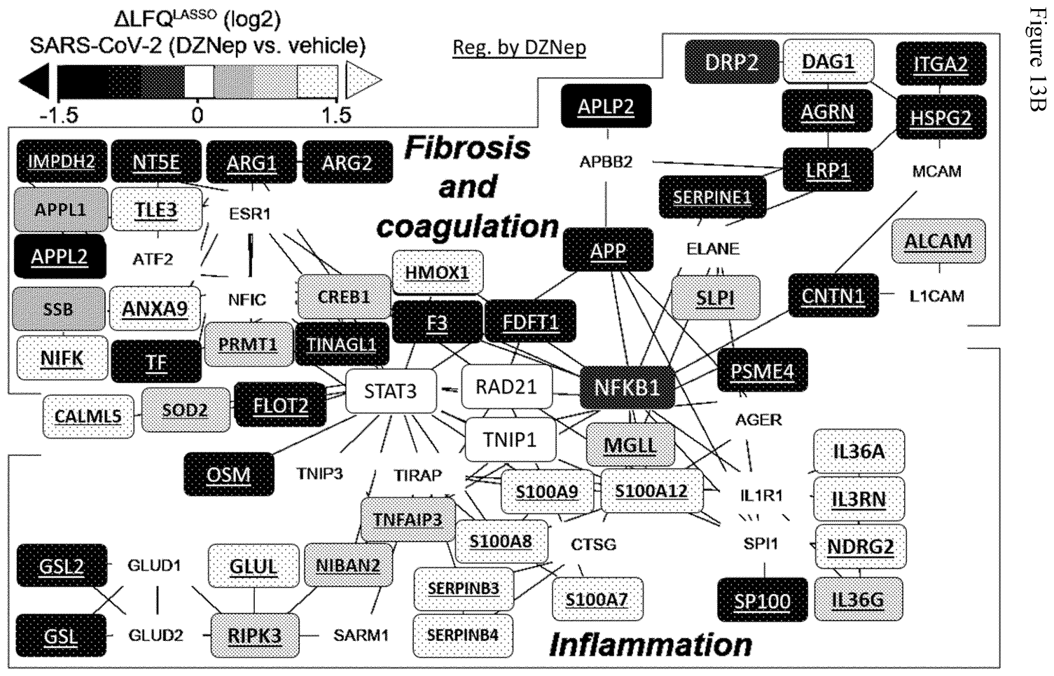

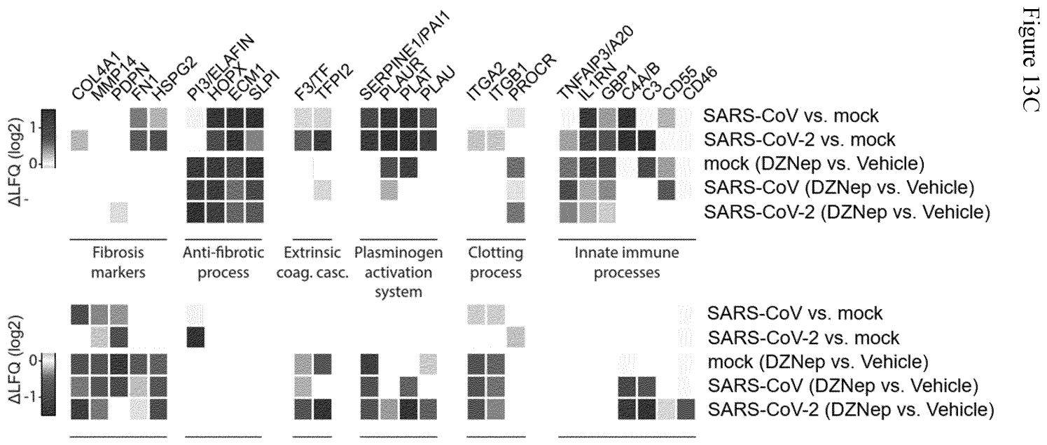

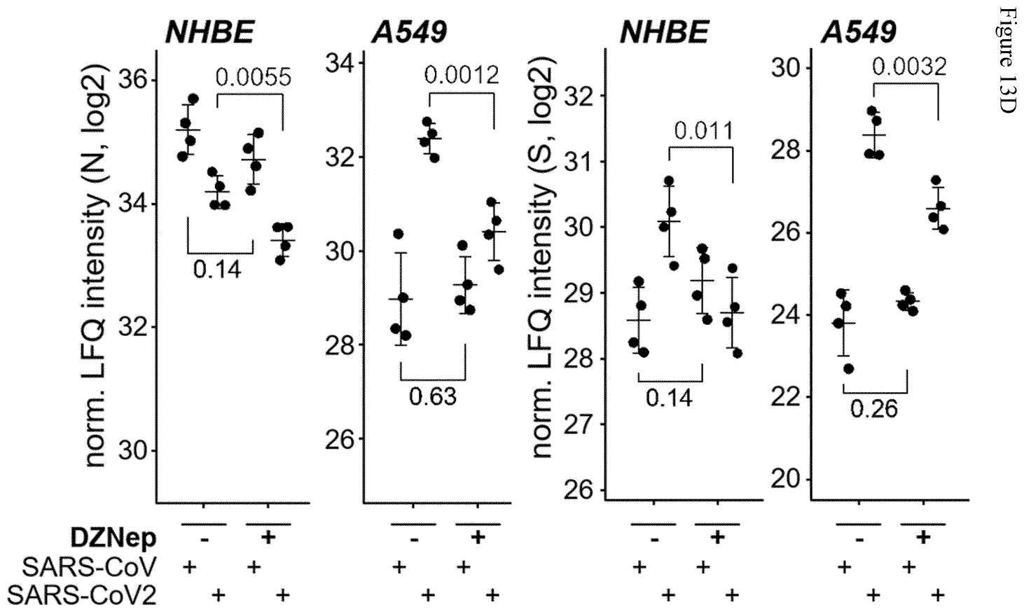

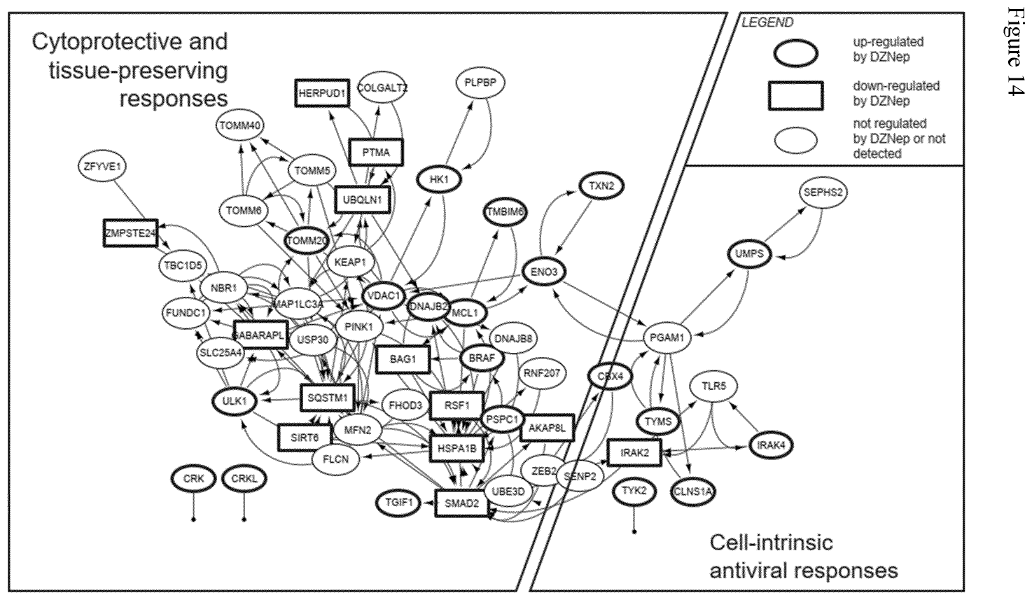

In order to investigate perturbation of cellular proteome upon DZNep treatment of mock-, SARS-CoV- and SARS-CoV-2-infected cells, we pre-treated A549-ACE2 cells or primary human bronchial epithelial cells (NHBEs) for 6 h with 0.75 μM (A549-ACE2) or 1.5 μM (NHBEs) DZNep and infected them with SARS-CoV and SARS-CoV-2 or mock. 24 (A549-ACE2) or 36 (NHBEs) hours post infection, we analysed proteome changes using mass spectrometry (LC-MS/MS). Upon examining expression of viral proteins in A549-ACE2 cells, we observed clear reduction in case of SARS-CoV-2 treated cells but not SARS-CoV treated cells (FIG. 13A). In order to examine the host protein expression changes on the system level in A549-ACE2 cells, we performed network diffusion analysis using proteins, differentially expressed upon DZNep treatment of SARS-CoV, SARS-CoV-2 or mock infected cells. In line with previous art, we observe protein networks associated to cytoprotective and tissue-preserving responses significantly enriched in the resulting network, indicating a strong potential for treatment of organ damage and tissue fibrosis, including lung fibrosis, by SAM-cycle component inhibitors such as DZNep (FIG. 14). In addition, we observe protein networks associated to cell-intrinsic antiviral responses, indicating a strong potential for treatment inducing secondary anti-viral effects in a subject (FIG. 14).

In order to explore the host processes, perturbed by DZNep in NHBEs, we performed network diffusion analysis (described in materials and methods) on proteins, significantly regulated by DZNep in SARS-CoV and SARS-CoV-2 infected conditions. This approach allows to highlight host proteins and pathways, which are functionally connected to the proteins of interest. Among the significantly enriched subnetworks was a cluster of genes functionally interacting with STAT3 and NFKB1 (FIG. 13B). In particular, this cluster can be subdivided into two distinct parts containing proteins related to biological processes governing fibrosis and blood coagulation, and inflammation (FIG. 13B). These findings led us to explore mentioned processes in more detail. Most notably, we observed that DZNep treatment of NHBE cells led to a reduction of pulmonary fibrosis biomarkers (e.g. COL4A1, MMP14 and SERPINE1; FIG. 13C, bottom panel) and up-regulation of factors counteracting fibrotic processes (e.g. ELAFIN, SLPI and ECM1; FIG. 13C upper panel). Furthermore, it led to reduction of factors of extrinsic coagulation cascade (e.g. F3 and TFPI2) and plasminogen activation system (e.g. PAI1, PLAT, PLAU) (FIG. 13C, bottom panel). Interestingly, we also observed DZNep-dependent changes in abundance of innate immunity related factors (e.g. IL1RN, C3 and TNFAIP3/A20) (FIG. 13C). In line with our previous findings, we show DZNep-dependent inhibition of SARS-CoV-2 but not of SARS-CoV as determined by abundance changes of NP and spike (S) (FIG. 13D).

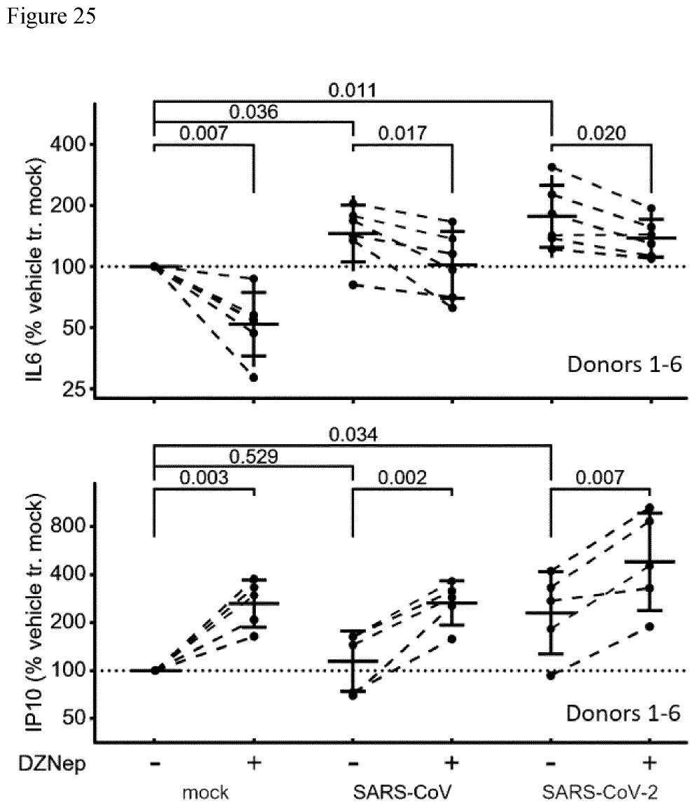

These findings prompted us to explore the impact of DZNep treatment on cell-intrinsic immunity ex vivo. We quantified secretion of the IRF3-dependent cytokine IP-10 and the NFKB-dependent cytokine IL-6 by ELISA 24 h post-infection of NHBEs with mock-, SARS-CoV or SARS-CoV-2 with optional pre-treatment with DZNep or its vehicle (FIG. 25). SARS-CoV and SARS-CoV-2 infection led to an increase in IL6 secretion. Interestingly, DZNep-treatment significantly reduced IL6 expression in all tested conditions (FIG. 25), which may be explained by its upregulation of TNFAIP3/A20. In contrast to IL6 and in line with MS-based observations concerning interferon induced proteins (e.g. IL1RN and GBP1), IP-10 secretion was enhanced after DZNep treatment (FIG. 25).

Taken together, we show that DZNep treatment of SARS-CoV-2 infected primary human NHBEs not only inhibits virus proliferation but also elicits anti-fibrotic effect and counteracts virus-induced deregulation of coagulation pathways. Furthermore, presented evidence indicate that DZNep treatment could balance the immune response to SARS-CoV-2 infection from NF-kB dominated inflammatory towards interferon dominated antiviral response.

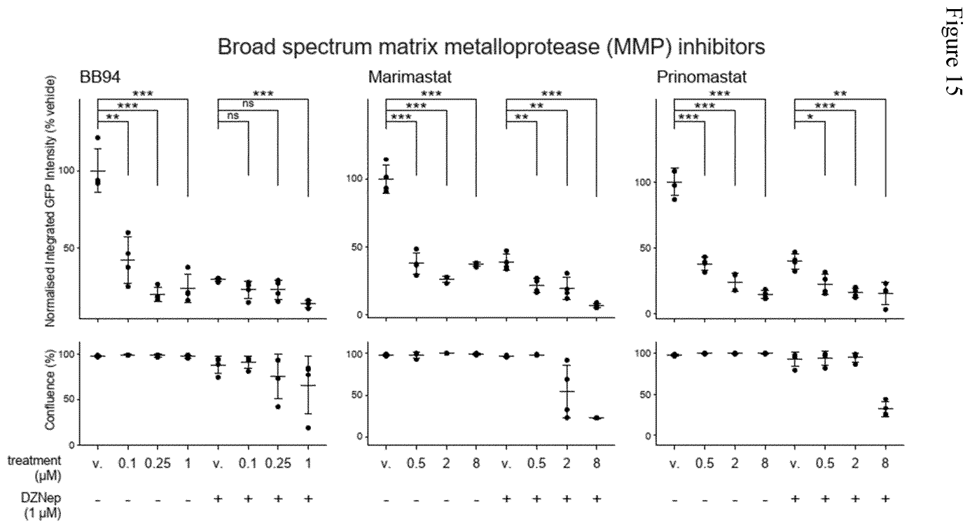

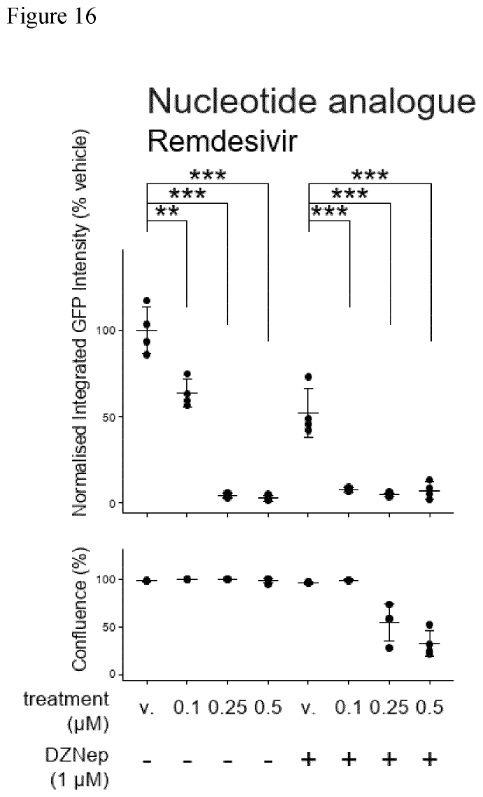

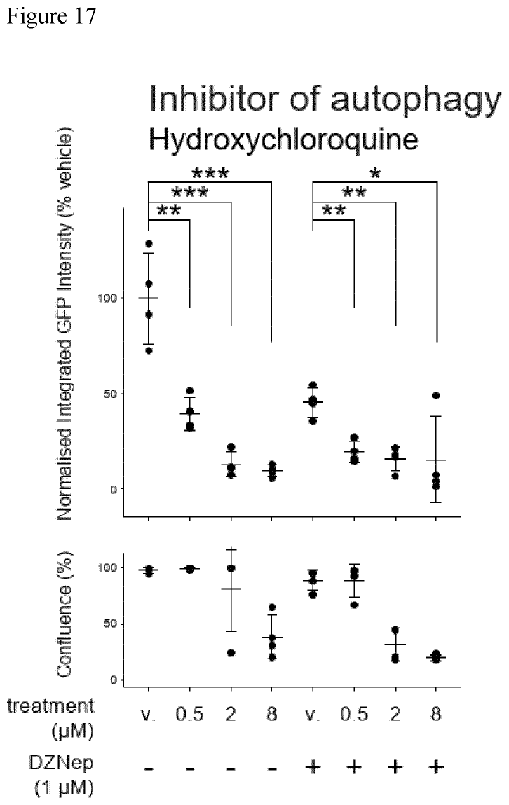

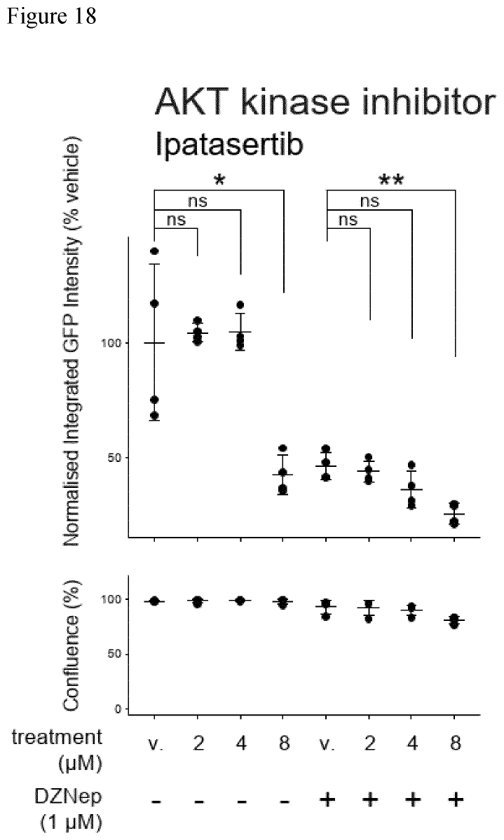

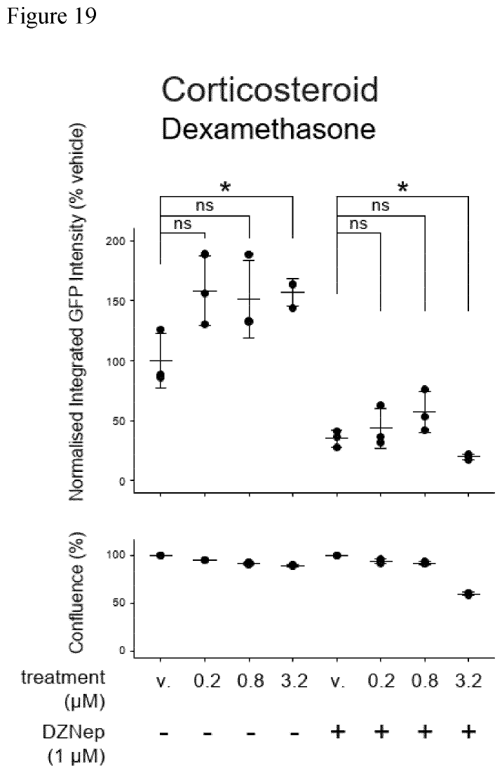

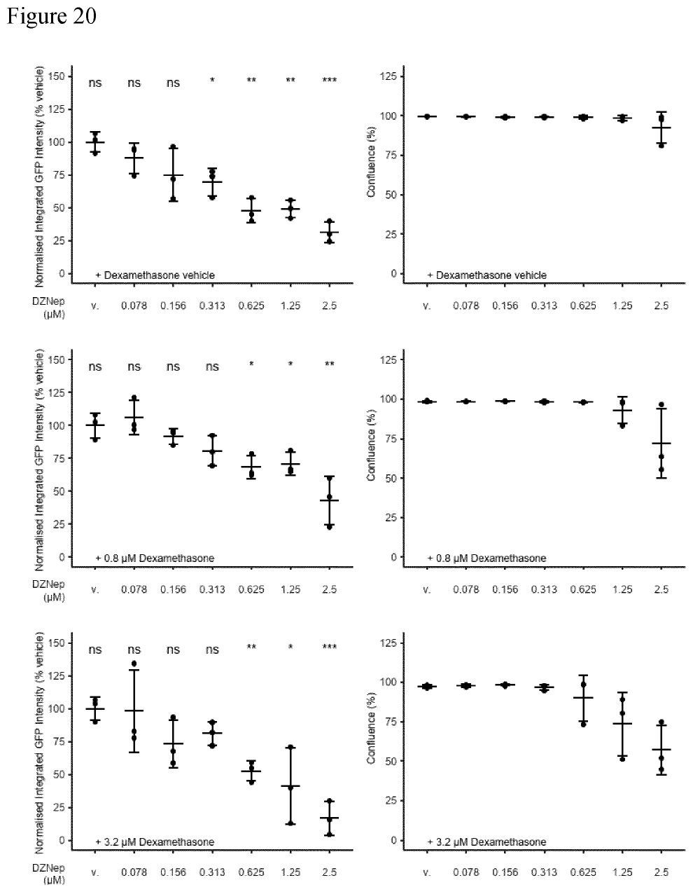

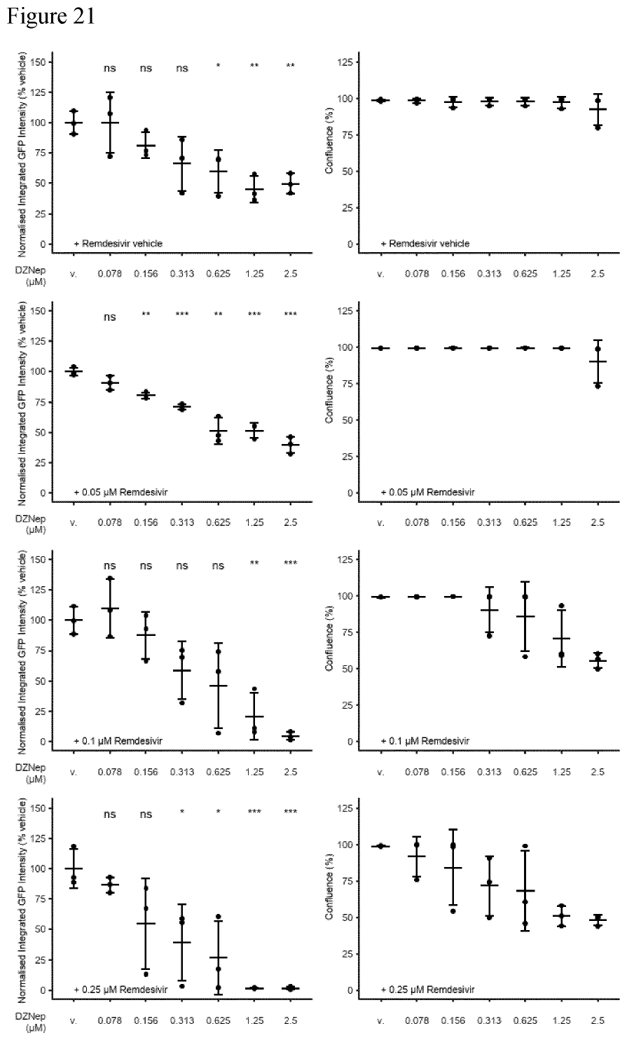

In order to investigate co-treatment potential of currently known antivirals active against SARS-CoV-2 with SAM-cycle inhibitors, we assessed the effect of DZNep co-treatment on the antiviral effects exhibited by a selection of representative compounds. Towards this, we pre-treated cells for 6 h with different concentrations of various treatments in combination with DZNep or vehicle. We show that DZNep does not negatively influence the antiviral efficacy of representative protease inhibitors (BB94, marimastat and prinomastat, FIG. 15), representative nucleotide analogue remdesivir (FIG. 16), representative inhibitor of autophagy (Hydroxychloroquine, FIG. 17), representative AKT inhibitor (ipatasertib, FIG. 18) and representative corticosteroid (dexamethasone, FIG. 19). To further investigate cotreatment potential and characterise synergistic antiviral effects of distinct known antivirals with a representative SAM-cycle inhibitor DZNep, we performed similar experiments utilising varying concentrations of DZNep. We show that for the case of dexamethasone the cotreatment results in more potent antiviral efficacy when higher concentrations of dexamethasone and DZNep are used in combination (FIG. 20). We show that for the case of remdesivir, the cotreatment is highly synergistic and that cotreatment is repressing the virus growth much more than individual treatments (FIG. 21). This speaks in favour of using SAM-cycle inhibitors in combinatorial therapies with existing drugs for treating SARS-CoV-2 infection and COVID-19.

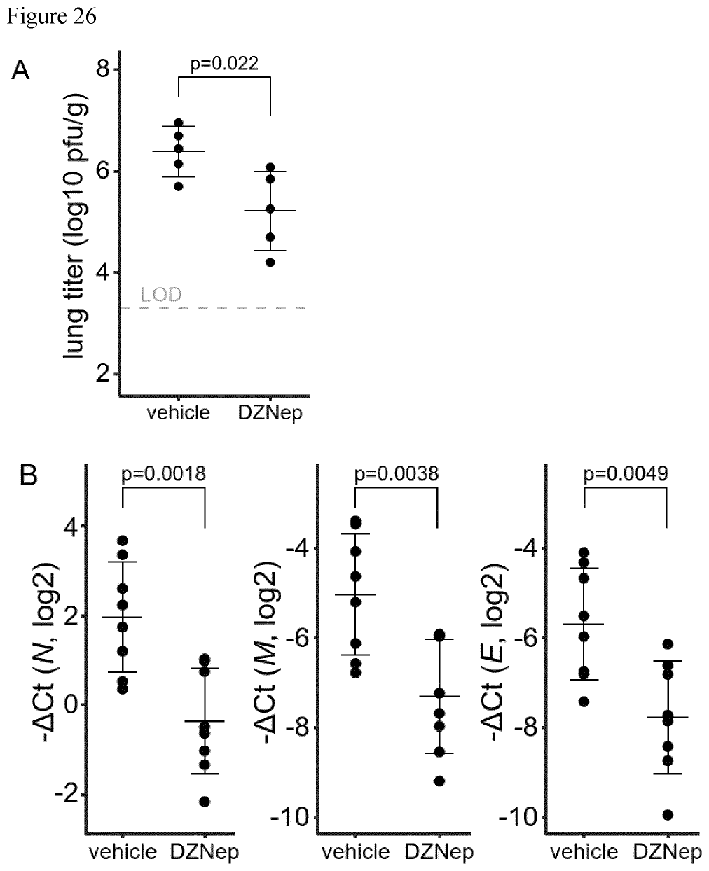

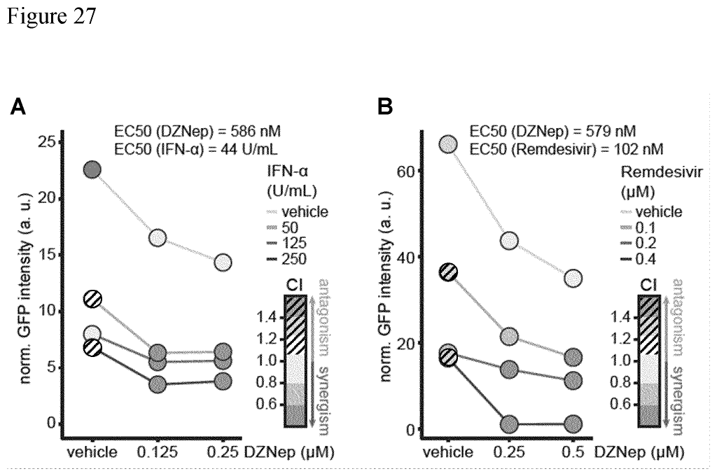

In order to test if DZNep treatment is antiviral against SARS-CoV-2 in vivo, we infected C57BL/6 mice with SARS-CoV-2 (B.1.351, also known as beta variant, 250 pfu, intranasal) and treated them at day zero and day one with DZNep (10 μg, intranasal) or vehicle control. At day two post-infection, approximating the early acute stage of infection, the animals were sacrificed and lungs were harvested. Lung infectious viral load was quantified by titration of lung homogenate supernatant on Vero E6 cells (FIG. 26A). We observed a significant reduction of the viral load in the lungs of DZNep treated animals relative to the control treatment (FIG. 26A). To corroborate these findings, lung RNA was isolated, reverse transcribed, and used for RT qPCR-based quantification of viral transcripts (FIG. 26B). We observed a significant DZNep-dependent reduction in abundance of viral transcripts for nucleoprotein (N), membrane protein (M) and envelope protein (E) (FIG. 26B), further corroborating that DZNep treatment elicits antiviral effect in murine SARS-CoV-2 infection model.

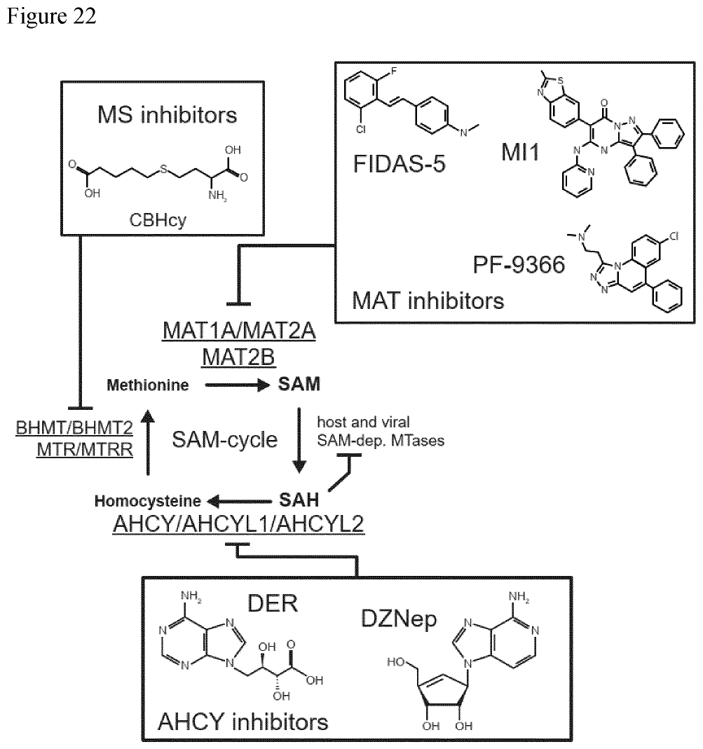

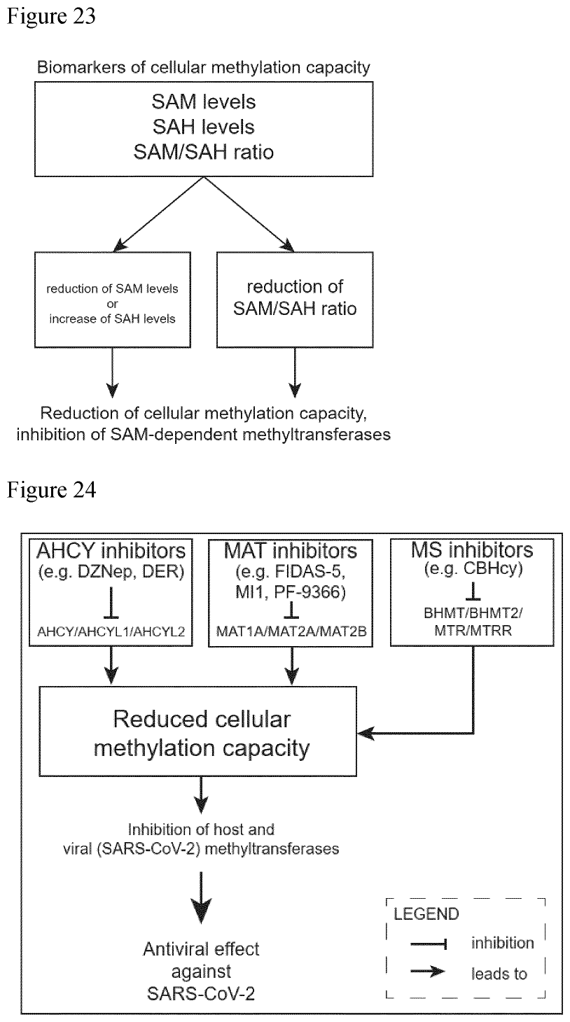

Overall, we show that inhibition of SAM-cycle components (FIG. 22), which leads to a reduction of cellular methylation capacity (FIG. 23, FIG. 24) and can be assessed by measuring known biomarkers SAM, SAH and the ratio between them, has a strong antiviral effect against SARS-CoV-2. The provided supporting data show that SAM-cycle component inhibitors are potential treatment options for SARS-CoV-2 infection and COVID-19, as well as underlying or COVID-19-caused organ damage and fibrosis.

Reagents, Viruses and Cell Lines

A549-ACE2 cells, Vero E6 cells and their respective culturing conditions were described previously⁶⁸. Primary normal human bronchial epithelial cells (NHBEs, Lonza, CC-2540) from genetically independent donors were cultured as described previously⁷¹. All cell lines were tested to be mycoplasma-free. RNA-isolation (Macherey-Nagel NucleoSpin RNA plus), reverse transcription (TaKaRa Bio PrimeScript RT with gDNA eraser) and RT-qPCR (Thermo-Fisher Scientific PowerUp SYBR green) were performed according to manufacturer protocol. For detection of protein abundance by western blotting and immunofluorescence, SARS-CoV/SARS-CoV N (Sino Biologicals, 40143-MM05), ACTB-HRP (Santa Cruz, sc-47778), Alexa Fluor 488 conjugated goat anti-mouse antibody (Abcam, ab150113) and anti-mouse HRP (Cell Signaling, 7076) antibodies were used. HRP and WB imaging was performed as described previously⁷². The following inhibitors were used: 3-deazaneplanocin A (InSolution EZH2 Inhibitor, DZNep—CAS 102052-95-9—Calbiochem, Sigma-Aldrich, 5060690001), D-eritadenine (Biomol, Cay21747-1), CBHcy (S-(4-Carboxybutyl)-D,L-homocysteine, BioTrend, AOB2142), tazemetostat (EPZ-6438, biomol, Cay16174-1), Remdesivir (Hölzel biotech, CS-0028115), Chloroquine (Chloroquine diphosphate salt, Sigma-Aldrich, C6628), Ribavirin (Sigma-Aldrich, R9644-10MG), dexamethasone (Sigma-Aldrich, D1756), BB-94 (Batimastat, Sigma-Aldrich, SML0041), Marimastat (Sigma-Aldrich, M2699), Prinomastat (Sigma-Aldrich, PZ0198), Ipatasertib (GDC-0068, 18412, Cayman chemical), FIDAS-5 (MAT2A Inhibitor II, FIDAS-5—Calbiochem, Sigma-Aldrich, 5041730001), MAT2A inhibitor 1 (Holzel Diagnostika, HY-112131) and PF-9366 (Holzel Diagnostika, HY-107778). The following sequences were used in multiplexed manner for cloning of gRNA into pLentiCRISPRv2 plasmid:

STAT1
(CACCGGGTGGCAAATGAAACATCAT, SEQ ID NO: 1; CACCGGAGGTCATGAAAACGGATGG,
SEQ ID NO: 2; CACCGCAGGAGGTCATGAAAACGGA, SEQ ID NO: 3),
NTC
(CACCGAACCGGATCGCCACGCGTCC, SEQ ID NO: 4; CACCGTCCGGAGCTTCTCCAGTCAA,
SEQ ID NO: 5; CACCGTGCAAAGTTCAGGGTAATGG, SEQ ID NO: 6),
AHCY
(CACCGTTTCCTCCCGTAGCCGACAT, SEQ ID NO:7; CACCGCCAGGCAGCCAGGCCGATGT,
SEQ ID NO: 8; CACCGTCCCGTAGCCGACATCGGCC, SEQ ID NO: 9),
MAT2A
(CACCGCTGGAATGATCCTTCTTGCT, SEQ ID NO: 10; CACCGTGGAATGATCCTTCTTGCTG,
SEQ ID NO: 11; CACCGTGCTGTTGACTACCAGAAAG, SEQ ID NO: 12),
EZH2
(CACCGCGGAAATCTTAAACCAAGAA, SEQ ID NO: 13; CACCGACCAAGAATGGAAACAGCGA,
SEQ ID NO: 14; CACCGACAGAAGTCAGGATGTGCAC, SEQ ID NO: 15).

Lentiviruses production, transduction of cells and antibiotic selection were performed as described previously⁷². In brief, A549-ACE2 cells were transduced using puromycin resistance carrying lentiviruses encoding Cas9 and gRNAs and grown for 3-5 days using medium, supplemented with 3 μg/mL puromycin, before being used for further experiments.

Virus Strains, Stock Preparation, Plaque Assay and In Vitro Infection

SARS-CoV-Frankfurt-1⁷³, SARS-CoV-2-MUC-IMB-1⁶⁷, SARS-CoV-2-B.1.1.7¹² and SARS-CoV-2-GFP^{67,68} strains, were produced as described previously⁶⁸.

A549-ACE2 cells were infected with either SARS-CoV-Frankfurt-1, SARS-CoV-2-MUC-IMB-1 or SARS-CoV-2-B.1.1.7 strains (MOI 3) for the subsequent experiments. At each time point, the samples were washed once with 1×PBS buffer and harvested in LBP (Macherey-Nagel) or 1×SSB lysis buffer (62.5 mM Tris HCl pH 6.8; 2% SDS; 10% glycerol; 50 mM DTT; 0.01% bromophenol blue) or freshly prepared SDC buffer (100 mM Tris HCl pH 8.5; 4% SDC) for RT-qPCR, western blot or LC-MS/MS analyses, respectively. The samples were heat-inactivated and frozen at −80° C. until further processing, as described in the following sections.

Viral Inhibition Assays

A549-ACE2 cells were seeded into 96-well plates in DMEM medium (10% FCS, 100 μg/ml Streptomycin, 100 IU/ml Penicillin) one day before infection. Six hours before infection, the medium was replaced with 125 μl of DMEM medium containing either the compound(s) of interest or their respective vehicle(s) as control. Infection was performed by adding 10 μl of SARS-CoV-2-GFP (MOI 3) per well and plates were placed in the IncuCyte S3 Live-Cell Analysis System where whole well real-time images of GFP and Phase channels were captured at regular time intervals. Cell viability was assessed as the cell confluence per well (Phase area). Virus growth was assessed as GFP integrated intensity normalized to cell confluence per well (GFP integrated intensity/Phase area) or GFP area normalized to cell confluence per well (GFP area/Phase area). Basic image analysis was performed using IncuCyte S3 Software (Essen Bioscience; version 2019B Rev2). Statistical analysis and visualisation was performed using R version 4.0.2.

Plaque Assays

Vero E6 cells were seeded into 24-well plates in DMEM medium (10% FCS, 100 μg/ml Streptomycin, 100 IU/ml Penicillin) one day before infection. Six hours before infection, the medium was replaced with 500 μl of DMEM medium containing either the compound(s) of interest or their respective vehicle(s) as control. Infection was performed by adding SARS-CoV-2 (MOI 0.1) to the well and the infection was allowed to progress for 48 hours. At that time, supernatants were harvested and frozen at −80° C. until further use.

Confluent monolayers of VeroE6 cells were infected with serial five-fold dilutions of virus supernatants (from 1:100 to 1:7812500) for 1 h at 37° C. The inoculum was removed and replaced with serum-free MEM (Gibco, Life Technologies) containing 0.5% carboxymethylcellulose (Sigma-Aldrich). Two days post-infection, cells were fixed for 20 minutes at room temperature with formaldehyde directly added to the medium to a final concentration of 5%. Fixed cells were washed extensively with PBS before staining with H2O containing 1% crystal violet and 10% ethanol for 20 minutes. After rinsing with PBS, the number of plaques was counted and the virus titer was calculated.

Viral Variant Comparison

For comparative analysis of antiviral treatment activity against SARS-CoV-2 strain MUC-IMB-1 and variant B.1.1.7, A549-ACE2 cells were seeded in 24-well plate one day before infection. Six hours before infection, the medium was replaced with 500 μl of DMEM medium containing either the compounds of interest or vehicle as a control. Infection was performed using MOI 3. Total cellular RNA was harvested and isolated using MACHEREY-NAGEL NucleoSpin RNA mini kit according to manufacturer instructions. Reverse transcription was performed using Takara PrimeScript RT reagent kit with gDNA eraser according to manufacturer instructions. RT-qPCR was performed using primers targeting SARS-CoV-2 N (fw: 5′-TTACAAACATTGGCCGCAAA-3′, SEQ ID NO: 16; rev: 5′-GCGCGACATTCCGAAGAA-3′, SEQ ID NO: 17) and human RPLP0 as housekeeper control (fw: 5′-GGATCTGCTGCATCTGCTTG-3′, SEQ ID NO: 18; rev: 5′-GCGACCTGGAAGTCCAACTA-3′, SEQ ID NO: 19) using PowerUp SYBR Green (Thermo Fisher, A25778) on QuantStudio 3 Real-Time PCR system (Thermo Fisher). Ct values, obtained using QuantStudio Design and Analysis Software v1.4.3, were averaged across technical replicates and -ΔCt values as a measure of gene expression were calculated as Ct(RPLP0)-Ct(N). Statistical analysis and visualisation was performed using R version 4.0.2.

Viral Protein Detection by Western Blotting

For detection of viral protein expression using Western blotting, A549-ACE2 cells were seeded in 24-well plate one day before infection. Six hours before infection, the medium was replaced with 500 μl of DMEM medium containing either the compounds of interest or vehicle as a control. Infection was performed with SARS-CoV-2 strain MUC-IMB-1 or SARS-CoV-2 strain Frankfurt 1 using MOI 3. At 24 hours post-infection, cells were lysed in SSB buffer (62.5 mM Tris HCl from 1M stock solution with pH 6.8, 2% SDS, 10% Glycerol, 50 mM DTT and 0.01% Bromophenol Blue in distilled water) and protein concentrations measured using Pierce 660-nm Protein Assay with an addition of Ionic detergent compatibility kit (Thermo Fischer Scientific) according to manufacturer instructions. Protein concentrations were equalised and up to 10 micrograms of proteins loaded in NuPAGE Bis-Tris, 1 mm, 4-12% gels (Thermo Fisher Scientific). Protein separation was performed according to gel manufacturer instructions and proteins transferred to 0.22 μm nitrocellulose membrane (1 h at 100V in 25 mM Trizma base, 0.192 Glycine, pH 8.3). The membranes were blocked for 1 hour in 5% non-fat milk in TBS-T buffer (0.25% Tween-20 in phosphate buffered saline solution) with gentle agitation. The following antibodies were used, diluted in 5% non-fat milk: anti-NP antibody for detection of SARS-CoV and SARS-CoV N (Sino Biologicals, 40143-MM05, 1:1000 dilution), ACTB-HRP (Santa Cruz, sc-47778, 1:2500 dilution), anti-mouse HRP (Cell Signaling, 7076, 1:2500 dilution). Western Lightning ECL Pro (PerkinElmer) was used for band detection according to manufacturer instructions. Normalisation of band signals was performed using Image Lab Software (Bio-Rad, version 6.0.1 build 34).

Viral Protein Detection by Immunofluorescence

For detection of viral protein expression using immunofluorescence, primary NHBE cells were cultured as described previously⁷¹. In brief, NHBEs were seeded in 96-well plate and grown until reaching 80% confluence. To avoid gene expression changes or influence on virus growth induced by growth factors in the BEGM medium (Lonza), cells were rested in basal medium (BEBM, Lonza) for 24 h before start of the experiment. The cells were pre-treated for 6 h with 3-deazaneplanocin A or vehicle as indicated and infected with SARS-CoV-2-MUC-IMB-1 or SARS-CoV-Frankfurt-1 at MOI 3. 24 h post-infection, cells were washed 3× with phosphate buffered saline (PBS), fixed for 15 minutes with 4% formaldehyde in PBS and blocked for 1 h using 4% BSA in PBS. The cells were further permeabilised using 0.1% Triton-X in 4% BSA PBS) for 15 min. Anti SARS-CoV and SARS-CoV2 N (Sino Biologicals, 40143-MM05) antibody was used in conjunction with Alexa Fluor 488 conjugated goat anti-mouse antibody (Abcam, ab150113) for detection of viral nucleoprotein in the cells (dilutions 1:1000 and 1:2500, respectively, in 4% BSA(PBS), sequential incubations with 5 washes with PBS in between). Thus stained cells were further washed 5 times with PBS and imaged using IncuCyte S3 Live-Cell Analysis System. Whole well images of GFP and Phase channels were captured. Cell viability and virus growth were assessed as the cell confluence per well (Phase area) and GFP integrated intensity normalized to cell confluence per well (GFP integrated intensity/Phase area) respectively using IncuCyte S3 Software (Essen Bioscience; version 2019B Rev2). Analysis and visualisation was performed using R version 4.0.2.

Quantification of Secreted Cytokines by ELISA

For detection of secreted cytokines, primary NHBE cells were cultured as described previously⁷¹. In brief, NHBEs were seeded in 12-well plate and grown until reaching 80% confluence. To avoid gene expression changes or influence on virus growth induced by growth factors in the BEGM medium (Lonza), cells were rested in basal medium (BEBM, Lonza) for 24 h before start of the experiment. The cells were pre-treated for 6 h with 3-deazaneplanocin A (0.75 μM) or vehicle (PBS) and infected with SARS-CoV-2-MUC-IMB-1 or SARS-CoV-Frankfurt-1 at MOI 3. 24 h post-infection, cell supernatant was harvested and frozen at −80° C. until further use.

For detection of human IL6 and IP10, commercially available ELISA kits were used (Human IL-6 ELISA Set, BD OptEIA, 555220; Human IP-10 ELISA Set, BD OptEIA, 550926) according to manufacturer instructions. Basal medium, used for NHBE culturing at time of treatment and infection, was used as blank control. Statistics were calculated using paired Student's two-sided t-test on log-transformed values between indicated conditions before donor-wise normalisation to vehicle treated mock controls.

Mass Spectrometry Sample Preparation and Analysis

For the determination of proteome changes in A549 cells, pre-treated for 6 h with vehicle (PBS) or 0.75 μM DZNep and infected with SARS-CoV-2 and SARS-CoV at MOI 3 for 24 h, cells were lysed in SDC lysis buffer (100 mM Tris HCl pH 8.5; 4% SDC). The following conditions were considered: vehicle-treated uninfected (3 replicates), DZNep-treated uninfected (4 replicates), vehicle-treated SARS-CoV-2 infected (4 replicates), DZNep-treated SARS-CoV-2 infected (4 replicates), vehicle-treated SARS-CoV infected (4 replicates), DZNep-treated SARS-CoV infected (4 replicates). For the determination of proteome changes in NHBEs, pre-treated for 6 h with vehicle (PBS) or 1.5 μM DZNep and infected with SARS-CoV-2 and SARS-CoV at MOI 3 for 24 h, cells were lysed in SDC lysis buffer (100 mM Tris HCl pH 8.5; 4% SDC). The following conditions were considered: vehicle-treated uninfected, DZNep-treated uninfected, vehicle-treated SARS-CoV-2 infected, DZNep-treated SARS-CoV-2 infected, vehicle-treated SARS-CoV infected, DZNep-treated SARS-CoV infected. Cells from 4 distinct donors were used. Sample preparation was performed as described previously⁶⁸. In brief, protein concentrations of cleared lysates were normalized and 50 μg used for further processing. To reduce and alkylate proteins, samples were incubated for 5 min at 45° C. with TCEP (10 mM) and CAA (40 mM). Samples were digested overnight at 37° C. using trypsin (1:100 w/w, enzyme/protein, Sigma-Aldrich) and LysC (1:100 w/w, enzyme/protein, Wako). Resulting peptide solutions were desalted using SDB-RPS StageTips (Empore). Samples were diluted with 1% TFA in isopropanol to a final volume of 200 μl and loaded onto StageTips, subsequently washed with 200 μl of 1% TFA in isopropanol and 200 μl 0.2% TFA/2% ACN. Peptides were eluted with 75 μl of 1.25% Ammonium hydroxide (NH₄OH) in 80% ACN and dried using a SpeedVac centrifuge (Eppendorf, Concentrator plus). They were resuspended in buffer A* (0.2% TFA/2% ACN) prior to LC-MS/MS analysis. Peptide concentrations were measured optically at 280 nm (Nanodrop 2000, Thermo Scientific) and subsequently equalized using buffer A*. 1 μg peptide was subjected to LC-MS/MS and protein groups quantified (MaxQuant version 1.6.10.43) with LFQ normalization (A549s) and without LFQ normalization (NHBEs) as described previously⁶⁸.

The analysis of MS data sets was performed using R version 4.0.2. LFQ values were log 2-transformed and protein groups only identified by site, reverse matches and potential contaminants excluded from the analysis. Additionally, protein groups quantified by a single peptide or not detected in all replicates of at least one condition were excluded from further analysis. In NHBE dataset, LFQ values were normalized for donor-specific effects on protein abundance. In short, the protein log 2-intensities were compared across conditions in a donor-wise manner, and systematic deviations across conditions subtracted in order to get normalized LFQ values.

The imputation of missing log 2-intensity values was done similarly to the method implemented in Perseus: the mean and the standard deviation of log 2-intensities were calculated for each dataset, and missing values were replaced by sampling from the normal distribution with the following parameters: 0.3*standard deviation, mean—1.8*standard deviation. In addition, effect scaling was performed using Gaussian generalized linear modeling approach (core function glm) to allow for quantitative comparison between virus infections and treatments in different contexts. In short, the following experiment design was used: norm. log 2-LFQ˜virus+virus:treatment, where virus refers to infection with mock, SARS-CoV or SARS-CoV-2, and treatment refers to vehicle or DZNep treatment. Median absolute values of significant effects (p<0.01) originating from virus and virus:treatment coefficients were calculated and divided by median of SARS-CoV-2 and mock:DZNep, respectively, resulting in coefficient 1+/−0.15 that were used in downstream analysis as coefficients in experimental design matrix.

The following experiment design was used for LASSO-based differential protein abundance analysis: LFQ˜virus+virus:treatment, where virus refers to infection with mock-, SARS-CoV or SARS-CoV-2, and treatment refers to vehicle or DZNep treatment. The following effects were thus estimated: effect of SARS-CoV infection, effect of SARS-CoV-2 infection, effect of DZNep treatment of mock-infected cells, effect of DZNep treatment of SARS-CoV infected cells and the effect of DZNep treatment of SARS-CoV-2 infected cells. The estimation of LASSO model parameters was performed using R package glmnet (version 4.0.2) with thresh=1e-28, maxit=1e7 and nfolds=11. The exact model coefficients and lambda value at cross-validation minimum (lambda.min) were extracted and used for p-value estimation by fixed-lambda LASSO inference using R package selectiveInference version 1.2.5. Default parameters were used with the following modifications: tol.beta=0.025, alpha=0.1, tailarea_rtol=0.1, tol.kkt=0.1 and bits=100. The bits parameter was set to 300 or 500 if the convergence was not reached. The sigma was explicitly estimated using function estimateSigma from the same package. No multiple hypothesis p-value correction was performed since that is handled by the choice of lambda. The following thresholds were applied to LASSO analysis results to identify statistically significant effects (log 2 fold-changes): p<10-5 and abs(log 2 fold change)>0.5 for the NHBE data, and p<10-4 and abs(log 2 fold change)>0.2 for A549 data. If a protein reached significance in one infected condition, or one treated condition, and not others, the significance thresholds for the other conditions were relaxed to: p<0.0 and abs(log 2 fold change)>0.2, in order to avoid over-estimating differences among similar infections or drug treatments.

Proteins, significantly changing in same direction (up- or down-regulated) upon DZNep treatment of SARS-CoV and SARS-CoV-2 infected NHBEs as determined by the above described analysis were used in network diffusion analysis. Network diffusion analysis was performed using ReactomeFI network v2019⁶¹. Random walk with restart kernel (R) was computed for this network in undirected manner, with restart probability of 0.4 according to the following equation: R=alpha*(I-(1-alpha)*W)⁻¹, where I is the identity matrix and W is the weight matrix computed asW=D⁻¹* A, where D is degree diagonal matrix and A is adjacency matrix for ReactomeFI graph. The diagonal values of the R matrix, representing restart- and feedback-flows, were excluded from subsequent analysis and set to 0. The significant hits from MS-data analysis were mapped to genes in the ReactomeFI network by matching gene names or their synonyms (from the biomaRt_hsapiens gene ensemble dataset) with the gene names in ReactomeFI. Nodes with significant flows originating from nodes representing hits in individual analyses were estimated using a randomization based approach. All hits and non-hits of the analysis were attributed equal weight (1 and 0, respectively) in subsequent statistical analysis. Flows to all nodes in the network were computed by multiplying the R matrix with the vector of hits described above. Furthermore, nodes in the network were assigned to 8 bins of approximately equal size according to the node degree. The same procedure of calculating inbound flows to all network nodes was repeated for 2500 iterations, each time using the same number of randomly selected decoy hits from sets of nodes with 1 bin higher node degree (on per-hit basis). The P-values describing the significance of functional connectivity to input hits were computed for each node according to the following formula: p=N(iteration with equal or higher inbound flux)/N(iterations). For visualization purposes, the ReactomeFI network was filtered for nodes that were either representing input proteins or proteins with p-values below 0.005 and further trimmed by removing non-hit nodes with degree equal to 1.

In Vivo Experiments

8 to 10 weeks-old C57BL/6J mice were purchased from Charles River Laboratories. Mice were anesthetized with 90 mg/kg Ketamine (WDT) and 9 mg/kg Xylazine (Serumwerk Bernburg AG). Mice were inoculated intranasally with 2.5×10² pfu of SARS-CoV-2 beta variant (also known as B.1.351). Infected mice were intranasally treated with 10 μg of DZNep at 30-60 minutes and 24 hours post infection. All animal experiments using SARS-CoV-2 were performed in a biosafety level 3 facility at University Hospital Bonn according to institutional and governmental guidelines of animal welfare (animal experiment application number 81-02.04.2019.A247).

Quantification of Virus Transcripts by RT-qPCR

At 2 days post infection, lungs of infected mice were harvested and homogenized in TRlzol (Invitrogen) using gentle MACS Octo Dissociator (Miltenyi Biotec). RNA was extracted from the homogenates following the manufacturer's protocol. cDNA was generated using High-Capacity cDNA Reverse Transcription Kit (Applied Biosystems). To quantify the viral RNA, real-time quantitative PCR was performed by Step One Plus Real-Time PCR System (Applied Biosystems) using Fast SYBR Green Master Mix (Applied Biosystems) and TaqMan Fast Advanced Master Mix (Applied Biosystems) (for transcripts M, Eand 18s rRNA), and by QuantStudio 3 Real-Time PCR system (Thermo Fisher) using PowerUp SYBR Green (Thermo Fisher) (for transcripts Nand Actb). RT-qPCR primers were designed for SARS-CoV-2 genes as below: 5′-TGTGACATCAAGGACCTGCC-3′; 5′-CTGAGTCACCTGCTACACGC-3′ for SARS-CoV-2 Mand 5′-ACAGGTACGTTAATAGTTAATAGCGT-3′; 5′-ATATTGCAGCAGTACGCACACA-3′ for SARS-CoV-2 E and 5′-TTACAAACATTGGCCGCAAA-3′; 5′-GCGCGACATTCCGAAGAA-3′ for SARS-CoV-2 N Levels of viral transcripts M and E were normalized with 18s rRNA levels using the TaqMan probe for eukaryotic 18s rRNA (Hs99999901_s1, Applied Biosystems). Levels of viral transcript N were normalized with Actb levels (RT-qPCR primers: 5′-CTCTGGCTCCTAGCACCATGAAGA-3′; 5′-GTAAAACGCAGCTCAGTAACAGTCCG-3′).

Quantification of Viral Load by Plaque Assay

30 mg of lungs were collected from infected mice at 2 days post-infection. Lungs were homogenized in 300 μl of PBS using Tissue Grinder Mixy Professional (NIPPON Genetics EUROPE, NG010). Homogenates were cleared by centrifugation twice (1500 rpm, 5 min, 4° C. and 15000 rpm, 5 min, 4° C.) and the supernatants were stored at −80° C. until further processing. The viral titers were determined by plaque assay using Vero E6 cells in the following manner. Confluent monolayers of VeroE6 cells were infected with 50 μl of serial ten-fold dilutions of virus supernatants (from 1:10 to 1:10000) for 1 h at 37° C. The inoculum was removed and replaced with serum-free MEM (Gibco, Life Technologies) containing 0.5% carboxymethylcellulose (Sigma-Aldrich). Two days post-infection, cells were fixed for 20 minutes at room temperature with formaldehyde directly added to the medium to a final concentration of 5%. Fixed cells were washed extensively with PBS before staining with H2O containing 1% crystal violet and 10% ethanol for 20 minutes. After rinsing with PBS, the number of plaques was counted and the virus titer was calculated.

The invention further refers to the following embodiments:

Embodiment 1. Inhibitor of at least one S-adenosylmethionine (SAM) cycle enzyme for use in preventing or treating coronavirus disease 2019 (COVID-19) in a subject, or for use in preventing or treating infection with severe acute respiratory syndrome coronavirus-2 (SARS-CoV-2) in a subject, wherein the at least one SAM cycle enzyme is selected from the group consisting of methionine adenosyltransferase, betaine-homocysteine methyltransferase, methionine synthase, methionine synthase reductase and S-adenosylhomocysteine hydrolase.

Embodiment 2. Inhibitor of at least one S-adenosylmethionine (SAM) cycle enzyme for use in preventing or treating lung fibrosis, interstitial pneumonia, acute lung injury (ALI), acute respiratory distress syndrome (ARDS), kidney injury, such as proteinuria and acute kidney injury, and vasculopathy.

Embodiment 3. Inhibitor for use of embodiment 1, wherein the at least one SAM cycle enzyme is selected from the group consisting of methionine adenosyltransferase and S-adenosylhomocysteine hydrolase.

Embodiment 4. Inhibitor for use of embodiment 3, wherein the methionine adenosyltransferase is methionine adenosyltransferase 1A (MAT1A) and/or methionine adenosyltransferase 2A (MAT2A) and/or methionine adenosyltransferase 2B MAT2B, preferably MAT2A.

Embodiment 5. Inhibitor for use of any one of the preceding embodiments, wherein inhibition of the at least one SAM cycle enzyme leads to decrease of the SAM concentration and/or the increase of S-adenosylhomocysteine (SAH) concentration and/or decrease of the ratio of SAM/SAH.

Embodiment 6. Inhibitor for use of any one of the preceding embodiments, wherein the AHCY inhibitor is selected from the group of carbocyclic nucleoside analogues and N9-alkylated adenine or pharmaceutical acceptable salts thereof.

Embodiment 7. Inhibitor for use of any one of the preceding embodiments, wherein the AHCY inhibitor is selected from the group consisting of D-eritadenine (DER) and 3-deazaneplanocin A (DZNep) or pharmaceutical acceptable salts thereof.

Embodiment 8. Inhibitor for use of any one of the preceding embodiments, wherein the AHCY inhibitor is DZNep or a pharmaceutical acceptable salt thereof.

Embodiment 9. Inhibitor for use of any one of the preceding embodiments, wherein the inhibitor inhibiting methionine adenosyltransferase is a fluorinated N,N-dialkylaminostilbene or pharmaceutical acceptable salts thereof.

Embodiment 10. Inhibitor for use of embodiment 9, wherein the fluorinated N,N-dialkylaminostilbene agents is (E)-4-(2-chloro-6-fluorostyryl)-N-methylaniline (FIDAS-5) or pharmaceutical acceptable salt thereof.

Embodiment 11. Inhibitor of embodiment 4, wherein the inhibitor inhibiting MAT2A is selected from the group consisting of FIDAS-5, MAT2A inhibitor 1 and PF-9366.

Embodiment 12. Inhibitor for use of any one of the preceding embodiments, wherein the inhibitor further inhibits also enhancer of zeste homolog 2 (EZH2).

Embodiment 13. Inhibitor for use of embodiment 12, wherein the inhibitor inhibits AHCY and EZH2.

Embodiment 14. Inhibitor for use according to any one of the preceding embodiments, wherein treating or preventing COVID-19 comprises preventing or treating lung fibrosis, interstitial pneumonia, acute lung injury (ALI), acute respiratory distress syndrome (ARDS), kidney injury, such as proteinuria and acute kidney injury, and vasculopathy.

Embodiment 15. Inhibitor for use according to any one of the preceding embodiments, wherein the subject suffers from fibrosis.

Embodiment 16. Inhibitor for use according to embodiment 15, wherein the subject suffers from lung fibrosis.

Embodiment 17. Inhibitor for use according to any one of the preceding embodiments, wherein preventing or treating COVID-19 comprises preventing or treating lung fibrosis caused by COVID-19.

Embodiment 18. Inhibitor for use according to any one of the preceding embodiments, wherein the subject is an immune deficient patient, preferably a patient suffering from type I interferon deficiency.

Embodiment 19. Inhibitor for use according to any one of the preceding embodiments, wherein a combination of at least two inhibitors inhibiting two different SAM cycle enzymes is administered.

Embodiment 20. Inhibitor for use according to any one of the preceding embodiments, wherein DZNep and FIDAS-5 are administered in combination.

Embodiment 21. Inhibitor for use according to any one of the preceding embodiments, wherein the inhibitor is administered in combination with a further therapeutic ingredient.

Embodiment 22. A pharmaceutical composition comprising the inhibitor according to any one of the preceding embodiments together with a pharmaceutically acceptable carrier and an optionally further therapeutic ingredient for use in preventing or treating COVID-19 in a subject, or for use in preventing or treating SARS-CoV-2 infection in a subject.

Embodiment 23. Inhibitor for use of embodiment 21 or composition for use of embodiment 22, wherein the further therapeutic ingredient is selected from the group consisting of protease inhibitors nucleotide analogues, inhibitors of autophagy, AKT kinase inhibitor, corticosteroids or interferons.

Embodiment 24. Inhibitor or composition for use according to embodiment 23, wherein the protease inhibitor is a broad spectrum matrix metalloprotease (MMP) inhibitor or serine protease inhibitor.

Embodiment 25. Inhibitor or composition for use according to embodiment 24, wherein MMP inhibitor is selected from the group consisting of BB94, marimastat, prionomastat.

Embodiment 26. Inhibitor or composition for use according to embodiment 24, wherein the serine protease inhibitor is camostat.

Embodiment 27. Inhibitor for use of embodiment 21 or composition for use of embodiment 22, wherein the further therapeutic ingredient is selected from the group consisting of BB94, marimastat, prinomastat, remdesivir, hydroxychloroquine, ipatasertib, camostat, dexamethasone and type I interferon.

Embodiment 28. Inhibitor for use of embodiment 27 or composition for used of embodiment 27, wherein the type I interferon is IFN-α.

Embodiment 29. Inhibitor for use or composition for use of embodiment 28, wherein the further therapeutic ingredient is selected from the group consisting of remdesivir and dexamethasone, preferably remdesivir.

Embodiment 30. Inhibitor of at least one S-adenosylmethionine (SAM) cycle enzyme for use in reducing SARS-CoV-2 viral transcripts in the respiratory system of a subject infected with SARS-CoV-2, wherein the at least one SAM cycle enzyme is selected from the group consisting of methionine adenosyltransferase, betaine-homocysteine methyltransferase, methionine synthase, methionine synthase reductase and S-adenosylhomocysteine hydrolase.

Embodiment 31. Inhibitor of at least one S-adenosylmethionine (SAM) cycle enzyme for use in inhibiting virus replication in the respiratory system of a subject infected with SARS-CoV-2, wherein the at least one SAM cycle enzyme is selected from the group consisting of methionine adenosyltransferase, betaine-homocysteine methyltransferase, methionine synthase, methionine synthase reductase and S-adenosylhomocysteine hydrolase.

Embodiment 32. Inhibitor for use of any one of embodiments 30 and 31, wherein the respiratory system comprises one or more of lungs, nose, nasopharynx and throat, preferably, wherein the respiratory system is lungs.

Embodiment 33. Inhibitor for use of any one of embodiments 30 to 32, wherein the SAM cycle enzyme inhibitor is DZNep or a pharmaceutical acceptable salt thereof.

### REFERENCE LIST

1. George, P. M., Wells, A. U. & Jenkins, R. G. Pulmonary fibrosis and COVID-19: the potential role for antifibrotic therapy. Lancet. Respir. Med 8, 807-815 (2020).
2. Ronco, C., Reis, T. & Husain-Syed, F. Management of acute kidney injury in patients with COVID-19. Lancet. Respir Med 8, 738-742 (2020).
3. O'Sullivan, J. M., Gonagle, D. M., Ward, S. E., Preston, R. J. S. & O'Donnell, J. S. Endothelial cells orchestrate COVID-19 coagulopathy. Lancet. Haematol 7, e553-e555 (2020).
4. Bai, J. et al. Identification of a natural inhibitor of methionine adenosyltransferase 2A regulating one-carbon metabolism in keratinocytes. EBioMedicine 39, 575-590 (2019).
5. Collinsova, M., Strakova, J., Jiracek, J. & Garrow, T. A. Inhibition of betaine-homocysteine S-methyltransferase causes hyperhomocysteinemia in mice. J. Nutr. 136, 1493-7 (2006).
6. Glazer, R. I. et al 3-Deazaneplanocin: a new and potent inhibitor of S-adenosylhomocysteine hydrolase and its effects on human promyelocytic leukemia cell line HL-60. Biochem. Biophys. Res. Commun. 135, 688-94 (1986).
7. Aury-Landas, J. et al. The Antitumoral Effect of the S-Adenosylhomocysteine Hydrolase Inhibitor, 3-Deazaneplanocin A, is Independent of EZH2 but is Correlated with EGFR Downregulation in Chondrosarcomas. Cell. Physiol Biochem. 53, 731-745 (2019).
8. Belužić, L. et al. Knock-down of AHCY and depletion of adenosine induces DNA damage and cell cycle arrest. Sci Rep. 8, 14012 (2018).
9. Baric, I. et al. S-adenosylhomocysteine hydrolase deficiency in a human: a genetic disorder of methionine metabolism. Proc. Natl. Acad. Sci. U S. A. 101, 4234-9 (2004).
10. Uchiyama, N. et al Identification of AHCY inhibitors using novel high-throughput mass spectrometry. Biochem. Biophys. Res. Commun. 491, 1-7 (2017).
11. Zhu, N. et al. A Novel Coronavirus from Patients with Pneumonia in China, 2019. N. Engl. J. Med 382, 727-733 (2020).
12. Coronaviridae Study Group of the International Committee on Taxonomy of Viruses. The species Severe acute respiratory syndrome-related coronavirus: classifying 2019-nCoV and naming it SARS-CoV-2. Nat Microbiol 5, 536-544 (2020).
13. Zhou, J. et al H19 lncRNA alters DNA methylation genome wide by regulating S-adenosylhomocysteine hydrolase. Nat. Commun. 6, 10221 (2015).
14. Shu, S. et al. S-adenosylhomocysteine hydrolase is localized at the front of chemotaxing cells, suggesting a role for transmethylation during migration. Proc. Natl. Acad. Sci. U.S.A 103, 19788-93 (2006).
15. Guillerm, G., Muzard, M., Glapski, C., Pilard, S. & De Clercq, E. Inactivation of S-adenosyl-L-homocysteine hydrolase by 6′-cyano-5′,6′-didehydro-6′-deoxyhomoadenosine and 6′-chloro-6′-cyano-5′,6′-didehydro-6′-deoxyhomoadenosine. Antiviral and cytotoxic effects. J. Med Chem. 49, 1223-6 (2006).
16. Hermes, M., Osswald, H. & Kloor, D. Role of S-adenosylhomocysteine hydrolase in adenosine-induced apoptosis in HepG2 cells. Exp. Cell Res. 313, 264-83 (2007).
17. Ando, T. et al. Synthesis of 2-modified aristeromycins and their analogs as potent inhibitors against Plasmodium falciparum S-adenosyl-L-homocysteine hydrolase. Bioorg. Med Chem. 16, 3809-15 (2008).
18. Li, Q.-S. et al. Comparative kinetics of cofactor association and dissociation for the human and trypanosomal S-adenosylhomocysteine hydrolases. 1. Basic features of the association and dissociation processes. Biochemistry 46, 5798-809 (2007).
19. Wang, M. et al. Effects of ligand binding and oxidation on hinge-bending motions in S-adenosyl-L-homocysteine hydrolase. Biochemistry 45, 7778-86 (2006).
20. Wu, Q.-L. et al. Inhibition of S-adenosyl-L-homocysteine hydrolase induces immunosuppression. J. Pharmacol. Exp. Ther. 313, 705-11 (2005).
21. Wang, M. et al. Are L-adenosine and its derivatives substrates for S-adenosyl-L-homocysteine hydrolase? J. Med Chem. 48, 3649-53 (2005).
22. Wnuk, S. F. et al. Stereoselective synthesis of sugar-modified enyne analogues of adenosine and uridine. Interaction with S-adenosyl-L-homocysteine hydrolase and antiviral and cytotoxic effects. J. Med Chem. 47, 5251-7 (2004).
23. Kitade, Y. et al. 4′-Fluorinated carbocyclic nucleosides: synthesis and inhibitory activity against S-adenosyl-L-homocysteine hydrolase. Bioorg. Med Chem. 14, 5578-83 (2006).
24. Hao, W. et al. Characterization of human S-adenosyl-homocysteine hydrolase in vitro and identification of its potential inhibitors. J. Enzyme Inhib. Med Chem. 32, 1209-1215 (2017).
25. Nakanishi, M., Iwata, A., Yatome, C. & Kitade, Y. Purification and properties of recombinant Plasmodium falciparum S-adenosyl-L-homocysteine hydrolase. J. Biochem. 129, 101-5 (2001).
26. Guillerm, G., Guillerm, D., Vandenplas-Vitkowski, C., Glapski, C. & De Clercq, E. Inactivation of S-adenosyl-L-homocysteine hydrolase with novel 5′-thioadenosine derivatives. Antiviral effects. Bioorg. Med Chem. Lett. 13, 1649-52 (2003).
27. Yang, X., Yin, D., Wnuk, S. F., Robins, M. J. & Borchardt, R. T. Mechanisms of inactivation of human S-adenosylhomocysteine hydrolase by 5′,5′,6′,6′-tetradehydro-6′-deoxy-6′-halohomoadenosines. Biochemistry 39, 15234-41 (2000).
28. Fabianowska-Majewska, K., Duley, J. A. & Simmonds, H. A. Effects of novel anti-viral adenosine analogues on the activity of S-adenosylhomocysteine hydrolase from human liver. Biochem. Pharmacol 48, 897-903 (1994).
29. Wnuk, S. F. et al Anticancer and antiviral effects and inactivation of S-adenosyl-L-homocysteine hydrolase with 5′-carboxaldehydes and oximes synthesized from adenosine and sugar-modified analogues. J. Med Chem. 40, 1608-18 (1997).
30. Hershfield, M. S., Aiyar, V. N., Premakumar, R. & Small, W. C. S-Adenosylhomocysteine hydrolase from human placenta. Affinity purification and characterization. Biochem. J. 230, 43-52 (1985).
31. Nakao, A. et al. Discovery and structural analyses of S-adenosyl-L-homocysteine hydrolase inhibitors based on non-adenosine analogs. Bioorg. Med Chem. 23, 4952-4969 (2015).
32. Nakao, A., Suzuki, H., Ueno, H., Iwasaki, H. & Setsuta, T. Discovery of S-adenosyl-L-homocysteine hydrolase inhibitors based on non-adenosine analogs. Bioorg. Med Chem. Lett. 24, 4336-40 (2014).
33. Converso, A. et al. Adenosine analogue inhibitors of S-adenosylhomocysteine hydrolase. Bioorg. Med Chem. Lett. 24, 2737-40 (2014).
34. Lee, K. M. et al. X-ray crystal structure and binding mode analysis of human S-adenosylhomocysteine hydrolase complexed with novel mechanism-based inhibitors, haloneplanocin A analogues. J. Med Chem. 54, 930-8 (2011).
35. Park, Y. H., Choi, W. J., Tipnis, A. S., Lee, K. M. & Jeong, L. S. Truncated fluorocyclopentenyl pyrimidines as S-adenosylhomocysteine hydrolase inhibitors. Nucleosides. Nucleotides Nucleic Acids 28, 601-13 (2009).
36. Li, Q.-S. et al Evaluation of NAD(H) analogues as selective inhibitors for Trypanosoma cruzi S-adenosylhomocysteine hydrolase. Nucleosides. Nucleotides Nucleic Acids 28, 473-84 (2009).
37. Kojima, H., Kozaki, A., Iwata, M., Ando, T. & Kitade, Y. Synthesis of 3′,4′-epoxynoraristeromycin analogs for molecular labeling probe of S-adenosyl-1-homocysteine hydrolase. Bioorg. Med Chem. 16, 6575-9 (2008).
38. Gupta, A. et al. Extrapulmonary manifestations of COVID-19. Nat Med 26, 1017-1032 (2020).
39. Sviripa, V. M. et al. 2′,6′-Dihalostyrylanilines, pyridines, and pyrimidines for the inhibition of the catalytic subunit of methionine S-adenosyltransferase-2. J. Med Chem. 57, 6083-91 (2014).
40. Zhang, W. et al. Fluorinated N,N-dialkylaminostilbenes repress colon cancer by targeting methionine S-adenosyltransferase 2A. ACS Chem. Biol 8, 796-803 (2013).
41. Quinlan, C. L. et al. Targeting S-adenosylmethionine biosynthesis with a novel allosteric inhibitor of Mat2A. Nat. Chem. Biol. 13, 785-792 (2017).
42. Ramani, K. et al. Leptin's mitogenic effect in human liver cancer cells requires induction of both methionine adenosyltransferase 2A and 2beta. Hepatology 47, 521-31 (2008).
43. Lu, S. C., Martinez-Chantar, M. L. & Mato, J. M. Methionine adenosyltransferase and S-adenosylmethionine in alcoholic liver disease. J. Gastroenterol Hepatol 21 Suppl 3, 561-4 (2006).
44. Corrales, F. J. et al. Regulation of mammalian liver methionine adenosyltransferase. J. Nutr. 132, 23775-23815 (2002).
45. Kotb, M. & Kredich, N. M. S-Adenosylmethionine synthetase from human lymphocytes. Purification and characterization. J. Bio/l Chem. 260, 3923-30 (1985).
46. Tallan, H. H. & Cohen, P. A. Methionine adenosyltransferase: kinetic properties of human and rat liver enzymes. Biochem. Med 16, 234-50 (1976).
47. Skiba, W. E., Wells, M. S., Mangum, J. H. & Awad, W. M. Betaine-homocysteine S-methyltransferase (human). Methods Enzymol 143, 384-8 (1987).
48. Skiba, W. E., Taylor, M. P., Wells, M. S., Mangum, J. H. & Awad, W. M. Human hepatic methionine biosynthesis. Purification and characterization of betaine:homocysteine S-methyltransferase. J. Biol. Chem. 257, 14944-8 (1982).
49. Vanek, V. et al. Structure-activity study of new inhibitors of human betaine-homocysteine S-methyltransferase. J. Med Chem. 52, 3652-65 (2009).
50. Kořínek, M. et al. Quantification of homocysteine-related metabolites and the role of betaine-homocysteine S-methyltransferase in HepG2 cells. Biomed Chromatogr. 27, 111-21 (2013).
51. Mádková, J. et al. Double-headed sulfur-linked amino acids as first inhibitors for betaine-homocysteine S-methyltransferase 2. J. Med Chem. 55, 6822-31 (2012).
52. Pícha, J. et al. The development of a new class of inhibitors for betaine-homocysteine S-methyltransferase. Eur. J. Med Chem. 65, 256-75 (2013).
53. Awad, W. M., Whitney, P. L., Skiba, W. E., Mangum, J. H. & Wells, M. S. Evidence for direct methyl transfer in betaine: homocysteine S-methyl-transferase. J. Biol. Chem. 258, 12790-2 (1983).
54. Bose, N., Greenspan, P. & Momany, C. Expression of recombinant human betaine: homocysteine S-methyltransferase for x-ray crystallographic studies and further characterization of interaction with S-adenosylmethionine. Protein Expr. Pur 25, 73-80 (2002).
55. Miller, C. M., Szegedi, S. S. & Garrow, T. A. Conformation-dependent inactivation of human betaine-homocysteine S-methyltransferase by hydrogen peroxide in vitro. Biochem. J. 392, 443-8 (2005).
56. Collinsová. M. et al. Combining combinatorial chemistry and affinity chromatography: highly selective inhibitors of human betaine: homocysteine S-methyltransferase. Chem. Biol 10, 113-22 (2003).
57. Ou, X. et al. Inhibition of human betaine-homocysteine methyltransferase expression by S-adenosylmethionine and methylthioadenosine. Biochem. J. 401, 87-96 (2007).
58. Jiracek, J. et al. S-alkylated homocysteine derivatives: new inhibitors of human betaine-homocysteine S-methyltransferase. J. Med. Chem. 49, 3982-9 (2006).
59. Castro, C., Millian, N. S. & Garrow, T. A. Liver betaine-homocysteine S-methyltransferase activity undergoes a redox switch at the active site zinc. Arch. Biochem. Biophys. 472, 26-33 (2008).
60. Szegedi, S. S., Castro, C. C., Koutmos, M. & Garrow, T. A. Betaine-homocysteine S-methyltransferase-2 is an S-methylmethionine-homocysteine methyltransferase. J. Biol. Chem. 283, 8939-45 (2008).
61. Tang, C. et al. Two newly synthesized 5-methyltetrahydrofolate-like compounds inhibit methionine synthase activity accompanied by cell cycle arrest in G1/S phase and apoptosis in vitro. Anticancer Drugs 19, 697-704 (2008).
62. Deng, X. et al. Design, synthesis and activities of aziridine derivatives of N5-methyltetrahydrofolate against methionine synthase. Chem. Res. Chinese Univ. 31, 742-745 (2015).
63. Wolthers, K. R., Lou, X., Toogood, H. S., Leys, D. & Scrutton, N. S. Mechanism of coenzyme binding to human methionine synthase reductase revealed through the crystal structure of the FNR-like module and isothermal titration calorimetry. Biochemistry 46, 11833-44 (2007).
64. Meints, C. E., Gustafsson, F. S., Scrutton, N. S. & Wolthers, K. R. Tryptophan 697 modulates hydride and interflavin electron transfer in human methionine synthase reductase. Biochemistry 50, 11131-42 (2011).
65. Meints, C. E., Simtchouk, S. & Wolthers, K. R. Aromatic substitution of the FAD-shielding tryptophan reveals its differential role in regulating electron flux in methionine synthase reductase and cytochrome P450 reductase. FEBS J. 280, 1460-74 (2013).
66. Meints, C. E., Parke, S. M. & Wolthers, K. R. Proximal FAD histidine residue influences interflavin electron transfer in cytochrome P450 reductase and methionine synthase reductase. Arch. Biochem. Biophys. 547, 18-26 (2014).
67. Thi Nhu Thao, T. et al. Rapid reconstruction of SARS-CoV-2 using a synthetic genomics platform. Nature 582, 561-565 (2020).
68. Stukalov, A. et al. Multi-level proteomics reveals host-perturbation strategies of SARS-CoV-2 and SARS-CoV. bioRxiv 2020.06.17.156455 (2020) doi:10.1101/2020.06.17.156455.
69. Xiao, X. et al. EZH2 enhances the differentiation of fibroblasts into myofibroblasts in idiopathic pulmonary fibrosis. Physiol Rep. 4, (2016).
70. Barnard, D. L. et al. Evaluation of immunomodulators, interferons and known in vitro SARS-coV inhibitors for inhibition of SARS-coV replication in BALB/c mice. Antivir Chem. Chemother. 17, 275-84 (2006).
71. Zissler, U. M. et al. Interleukin-4 and interferon-γ orchestrate an epithelial polarization in the airways. Mucosal Immunol 9, 917-26 (2016).
72. Scaturro, P. et al. An orthogonal proteomic survey uncovers novel Zika virus host factors. Nature 561, 253-257 (2018).
73. Pfefferle, S. et al. Reverse genetic characterization of the natural genomic deletion in SARS-Coronavirus strain Frankfurt-1 open reading frame 7b reveals an attenuating function of the 7b protein in-vitro and in-vivo. Virol J. 6, 131 (2009).
74. Chou, T. C. & Talalay, P. Quantitative analysis of dose-effect relationships: the combined effects of multiple drugs or enzyme inhibitors. Adv. Enzyme Regul. 22, 27-55 (1984).

1. Inhibitor of at least one S-adenosylmethionine (SAM) cycle enzyme for use in preventing or treating coronavirus disease 2019 (COVID-19) in a subject, or for use in preventing or treating infection with severe acute respiratory syndrome coronavirus-2 (SARS-CoV-2) in a subject, wherein the at least one SAM cycle enzyme is selected from the group consisting of S-adenosylhomocysteine hydrolase, methionine adenosyltransferase, betaine-homocysteine methyltransferase, methionine synthase and methionine synthase reductase.

2. Inhibitor for use according to claim 1, wherein the inhibitor inhibits virus replication in the respiratory system of a subject infected with SARS-CoV-2.

3. Inhibitor for use of claims 1 or 2, wherein at least one SAM cycle enzyme is selected from the group consisting of S-adenosylhomocysteine hydrolase and methionine adenosyltransferase.

4. Inhibitor for use of any one of the preceding claims, wherein inhibition of the at least one SAM cycle enzyme leads to decrease of the SAM concentration and/or increase of the S-adenosylhomocysteine (SAH) concentration and/or decrease of the ratio of SAM/SAH.

5. Inhibitor for use of any one of the preceding claims, wherein the inhibitor inhibiting S-adenosylhomocysteine hydrolase inhibitor is selected from the group consisting of D-eritadenine (DER) and 3-deazaneplanocin A (DZNep) or pharmaceutical acceptable salts thereof.

6. Inhibitor for use of any one of the preceding claims, wherein the inhibitor inhibiting S-adenosylhomocysteine hydrolase is DZNep or a pharmaceutical acceptable salt thereof.

7. Inhibitor for use of any one of the preceding claims, wherein the inhibitor inhibiting methionine adenosyltransferase is a fluorinated N,N-dialkylaminostilbene or pharmaceutical acceptable salts thereof.

8. Inhibitor for use of claim 7, wherein the inhibitor inhibiting methionine adenosyltransferase is selected from the group consisting of FIDAS-5, MAT2A inhibitor 1 and PF-9366 or a pharmaceutical acceptable salt thereof.

9. Inhibitor for use according to any one of the preceding claims, wherein treating or preventing COVID-19 comprises preventing or treating lung fibrosis, interstitial pneumonia, acute lung injury (ALI), acute respiratory distress syndrome (ARDS), kidney injury, such as proteinuria and acute kidney injury, and vasculopathy.

10. Inhibitor for use according to any one of the preceding claims, wherein a combination of at least two inhibitors inhibiting two different SAM cycle enzymes is administered.

11. Inhibitor for use according to any one of the preceding claims, wherein DZNep and FIDAS-5 are administered in combination.

12. Inhibitor for use according to any one of the preceding claims, wherein the inhibitor is administered in combination with a further therapeutic ingredient.

13. A pharmaceutical composition comprising the inhibitor according to any one of the preceding claims together with a pharmaceutically acceptable carrier and an optionally further therapeutic ingredient for use in preventing or treating COVID-19 in a subject, or for use in preventing or treating SARS-CoV-2 infection in a subject.

14. Inhibitor for use of claim 12 or composition for use of claim 13, wherein the further therapeutic ingredient is selected from the group consisting of protease inhibitors, nucleotide analogues, inhibitors of autophagy, AKT kinase inhibitor, corticosteroids or interferons.

15. Inhibitor for use of claim 12 or composition for use of claim 13, wherein the further therapeutic ingredient is selected from the group consisting BB94, marimastat, prinomastat, remdesivir, hydroxychloroquine, ipatasertib, camostat, dexamethasone and type I interferon.

16. Inhibitor for use or composition for use of claim 15, wherein the further therapeutic ingredient is selected from the group consisting of remdesivir and dexamethasone, preferably remdesivir.

17. DZNep for use in preventing or treating coronavirus disease 2019 (COVID-19) in a subject, or for use in preventing or treating infection with severe acute respiratory syndrome coronavirus-2 (SARS-CoV-2) in a subject.

18. DZNep for use in inhibiting virus replication in the respiratory system of a subject infected with SARS-CoV-2.
